(19) United States
(12) Patent Application Publication
(10) Pub. No.: US 2024/0262853 A1
(43) Pub. Date: Aug. 8, 2024

(54) SELECTIVE SEPARATION OF HEXACHLOROPLATINATE(IV) DIANIONS BASED ON EXO-BINDING WITH CUCURBIT[6]URIL

(71) Applicant: Northwestern University, Evanston, IL (US)

(72) Inventors: James Fraser Stoddart, Evanston, IL (US); Yu Wang, Evanston, IL (US); Huang Wu, Evanston, IL (US)

(21) Appl. No.: 18/563,509

(22) PCT Filed: May 24, 2022
(86) PCT No.: PCT/US22/30741

§ 371 Date: Nov. 22, 2023

Related U.S. Application Data
(60) Provisional application No. 63/202,038, filed on May 24, 2021.

(51) Int. Cl. C07F 15/00; C07D 487/22; C22B 3/20; C22B 3/44; C22B 11/00

(52) U.S. Cl. CPC C07F 15/0006; C07D 487/22; C22B 3/205; C22B 3/44; C22B 11/048

(57) ABSTRACT

Disclosed herein are compounds, compositions, and methods for separating platinum halide dianions, such as [PtCl₆]²⁻, with a cubcurbituril, such as cucurbit[6]uril, is disclosed herein.

### CROSS-REFERENCE TO RELATED APPLICATIONS

This application claims priority to U.S. Patent Application Ser. No. 63/202,038, filed May 24, 2021, the contents of which are incorporated by reference in its entirety.

### FIELD OF THE INVENTION

The disclosed technology is generally directed to extraction of metals, such as platinum. More particularly the technology is directed to hexachloroplatinate(IV) dianions with a cucurbituril.

### BACKGROUND OF THE INVENTION

Anion recognition has become one of the most active areas for research in supramolecular chemistry, since anions play crucial roles in biological, environmental and materials sciences. Following its vigorous growth during the past several decades, anion recognition has spawned a number of applications, including anion extraction and separation, anion sensing, transmembrane transport, and organocatalysis. In this context, many well-crafted artificial macrocyclic anion-receptors, e.g., crown ethers, calixarenes, calix[4]pyrroles, cyanostars, bambus[6]urils, biotin[6]uril esters, cyclopeptides, and others have been synthesized, which exhibit highly specific recognition for particular anions. The [PtCl₆]²⁻ dianion, a stable platinum metalate, is a key intermediate in the platinum-mining process and an indispensable upstream product in the platinum-chemical industry. In recent years, the demand for platinum has been increasing steadily.

It has found a wide range of applications in chemical synthesis, jewelry manufacture, electronic fabrication, automotive exhaust gas treatment, and anticancer drug production. It follows that the development of molecular receptors, which are capable of recognizing and separating [PtCl₆]²⁻ dianions selectively, is significant to the recovery of platinum metal. There are only a handful of reports, however, on receptors for [PtCl₆]²⁻ dianions, including hydrazine-functionalized zeolites, p-diethylaminomethylthia-calix[4]arene, and tripodal ionophores, which either adsorb [PtCl₆]²⁻ dianions in their porous channels or bind them in their cavities. It is clear that there is a need to development of more efficient ways to probe the selective recognition and separation of [PtCl₆]²⁻ dianions and the like.

### BRIEF SUMMARY OF THE INVENTION

Disclosed herein are compounds, compositions, and methods for separating dianions, such as [PtCl₆]²⁻, with a cubcurbituril, such as cucurbit[6]uril, is disclosed herein. The presently disclosed technology may be used for extraction of metals, such as platinum. The use of the present technology allows for platinum extraction from spent vehicular three-way catalytic converters and other platinum-bearing metal waste. The present technology allows for selective recognition of [PtCl_{6]}²⁻ dianion dianions through noncovalent bonding interactions on the outer surface of CB[6], selective co-crystallization with [PtCl₆]²⁻ dianion with CB[6] even in the presence of [PdCl₄]²⁻ and [RhCl₆]³⁻ anions; co-precipitation within seconds at ambient conditions; and recovery of platinum from its mixtures in an environmentally friendly manner.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. (a) Platinum-recovery flow diagram. (b) Platinum-recovery flow diagram based on the cocrystals of CB[6]·H₂PtCl₆. Bolded arrows indicate the flow direction for the recovery of platinum. (c) Structural formula, ball-and-stick representation, and electrostatic potential map of CB[6]. (d) Visual display of the selective co-crystallization between CB[6] and H₂PtCl₆. (e) Raman spectra of CB[6], H₂PtCl₆, and the CB[6]·H₂PtCl₆ adduct. (f) Powder X-ray diffraction patterns of the CB[6]·H₂PtCl₆ adduct obtained by instantaneous co-crystallization, compared with simulated patterns derived from X-ray crystallographic data.

FIG. 2. (a, b) SEM Images of the CB[6]·H₂PtCl₆ microcrystals prepared by adding directly an equimolar amount of CB[6] to an aqueous H₂PtCl₆ solution. (c) SEM-EDS Maps of the CB[6]·H₂PtCl₆ adduct showing all the component elements well distributed within the microrods.

FIG. 3. (a) Ball-and-stick representation illustrating that every CB[6] molecule (F) is surrounded by six adjacent [PtCl₆]²⁻ dianions (1-6). (b) DFT Calculated binding energies between a CB[6] molecule (F) and six connected [PtCl₆]²⁻ dianions (1-6). (c) Ball-and-stick representation showing that every [PtCl₆]²⁻ dianion interacts with six CB[6] molecules, courtesy of [Pt—Cl . . . H—C]hydrogen bonding and [Pt—Cl . . . C═O] ion-dipole interactions. (d) Visualized intermolecular binding iso-surface between CB[6] and [PtCl₆]²⁻. H₂O Molecules are omitted for the sake of clarity.

FIG. 4. Solid-state superstructures (above) and calculated binding energies (below) between CB[6] and four different platinum group metals anions. (a) Crystal superstructure showing how a central [PtCl₆]²⁻ dianion (1) interacts with six adjacent CB[6] molecules (A-F) in the solid state, as well as calculated binding energies between a [PtCl₆]²⁻ dianion (1) and six connected CB[6] molecules (A-F). (b) Crystal superstructure showing how a central [PtCl₄]²⁻ dianion (1) interacts with five adjacent CB[6] (A-E) and three water molecules in the solid state, as well as calculated binding energies between a [PtCl₄]²⁻ dianion (1) and five connected CB[6] molecules (A-E). (c) Crystal superstructure showing how a central [PdCl₄]²⁻ dianion (1) interacts with five adjacent CB[6] (A-E) and two water molecules in the solid state, as well as calculated binding energies between a [PdCl₄]²⁻ dianion (1) and five connected CB[6] (A-E) molecules. (d) Crystal superstructure showing how a central [RhCl₆]³⁻ trianion (1) interacts with six adjacent CB[6] molecules (A-F) in the solid state, as well as calculated binding energies between a [RhCl₆]³⁻ trianion (1) and six connected CB[6] molecules (A-F). 1-X (X=A-F) representing the two interacting molecules defined in their crystal structures.

FIG. 5. (a) Schematic diagram of the selective recovery of platinum using CB[6]. (b) SEM-EDS Maps of the CB[6]·H₂PtCl₆ microcrystals obtained by adding CB[6] to an aqueous solution of equimolar amounts of [PtCl₆]²⁻, [PdCl₄]²⁻ and [RhCl₆]³⁻ anions. (c) Effect of changes in the concentration of the mixture on the selective separation of [PtCl₆]²⁻ dianions from a mixture of [PtCl₆]²⁻, [PdCl₄]²⁻ and [RhCl₆]³⁻ anions in aqueous solution. (d) XPS Spectra of CB[6]·H₂PtCl₆ microcrystals, and the microcrystals obtained by adding CB[6] to an aqueous mixture of [PtCl₆]²⁻, [PdCl₄]²⁻ and [RhCl₆]³⁻ anions, followed by washing with H₂O.

FIG. 6. Selective co-crystallization between CB[6] and H₂PtCl₆. When a 4 M HCl solution of CB[6] (1.0 mL, 10 mM) was added to aqueous solutions of H₂PtCl₆, (NH₄)₂PdCl₆, (NH₄)₂PdCl₄, (NH₄)₃RhCl₆ (1.0 mL, 10 mM), a yellow suspension formed exclusively between CB[6] and H₂PtCl₆.

FIG. 7. Panel (a) shows a photograph of CB[6]·H₂PtCl₆ cocrystals, obtained by adding CB[6] to an aqueous solution of [PtCl₆]²⁻, [PdCl₄]²⁻ and [RhCl₆]³⁻ anions, panel (b) shows a photograph of recovered platinum metal, obtained by reducing the cocrystals with N₂H₄·H₂O, and panel (c) shows a photograph of regenerated CB[6], obtained by precipitating with Me₂CO.

FIG. 8. Solid-state superstructure of the adduct formed between CB[6] molecules and [PtCl₄]²⁻ dianions, and NH₄⁺ cations are absent in the crystal lattice. Panel (a) shows ball-and-stick representation showing that CB[6] molecule interacts with three or four [PtCl₄]²⁻ dianions through [Pt—Cl . . . H—C] hydrogen-bonding, [Pt—Cl . . . C═O] ion-dipole and [Pt . . . H—C] interactions. Panel (b) shows ball-and-stick representation showing that every [PtCl₄]²⁻ dianion is surrounded by five adjacent CB[6] molecules. Panel (c) shows a trimer of CB[6] molecules is sustained by the intermolecular [C═O . . . H—C] hydrogen-bonding interactions. Panel (d) shows outer surface interaction between CB[6] trimers and [PtCl₄]²⁻ dianions. The H₂O molecules are omitted for the sake of clarity.

FIG. 9. Solid-state superstructure of the adduct formed between CB[6] molecules and [PdCl₄]²⁻ dianions, and NH₄⁺ cations are absent in the crystal lattice. Panel (a) shows ball-and-stick representation showing that CB[6] molecule interacts with three or four [PdCl₄]²⁻ dianions through [Pd—Cl . . . H—C] hydrogen-bonding, [Pd—Cl . . . C═O] ion-dipole and [Pd . . . H—C] interactions. Panel (b) shows ball-and-stick representation showing that every [PdCl₄]²⁻ dianion is surrounded by five adjacent CB[6] molecules. Panel (c) shows a trimer of CB[6] molecules is sustained by the intermolecular [C═O . . . H—C] hydrogen-bonding interactions. Panel (d) shows outer surface interaction between CB[6] trimers and [PdCl₄]²⁻. The H₂O molecules are omitted for the sake of clarity.

FIG. 10. Solid-state superstructure of the adduct formed between CB[6] molecules and [RhCl₆]³⁻ trianions, and NH₄⁺ cations are absent in the crystal lattice. Panel (a) shows ball-and-stick representation showing that every CB[6] molecule interacts with two [RhCl₆]³⁻ trianions through [Rh—Cl . . . H—C] hydrogen-bonding and [Rh—Cl . . . C═O] ion-dipole interactions, every [RhCl₆]³⁻ trianions disorder over two positions. Panel (b) shows ball-and-stick representation showing that every [RhCl₆]³⁻ trianion surrounded by six adjacent CB[6] molecules. Panel (c) shows outer surface interaction between CB[6] and [RhCl₆]³⁻. The H₂O molecules are omitted for the sake of clarity.

FIG. 11. SEM images of the CB[6]·H₂PtCl₆ microcrystals obtained by adding equimolar CB[6] to an aqueous solution of [PtCl₆]²⁻ dianions, illustrating in Panel (a) the rod-like microstructures, Panel (b) magnified solid and hollow microrods for the CB[6]·H₂PtCl₆ microcrystals.

FIG. 12. SEM images of the CB[6]·H₂PtCl₆ microcrystals obtained by adding CB[6] to a mixture solution of [PtCl₆]²⁻, [PdCl₄]²⁻ and [RhCl₆]³⁻ anions, illustrating in Panel (a) the rod-like microstructures, Panel (b) magnified hollow microrods, and Panels (c) and (d) flower-like aggregates of the CB[6]·H₂PtCl₆ microrods.

FIG. 13. Raman spectra of Panel (a) shows a Raman spectra of H₂PtCl₆, Panel (b) shows a Raman spectra of microcrystals from the mixture, obtained by adding CB[6] to an aqueous solution of [PtCl₆]²⁻, [PdCl₄]²⁻ and [RhCl₆]³⁻ anions, panel (c) show a Raman spectra of microcrystals of CB[6]·H₂PtCl₆, and Panel (d) shows a Raman spectra of CB[6].

FIG. 14. Powder X-ray diffraction patterns of Panel (a) microcrystals from the mixture, obtained by adding CB[6] to an aqueous solution of [PtCl₆]²⁻, [PdCl₄]²⁻ and [RhCl₆]³⁻ anions, Panel (b) microcrystals of CB[6]·H₂PtCl₆, and Panel (c) simulation, derived from the single-crystal X-ray crystallographic data of CB[6]·H₂PtCl₆ adducts.

FIG. 15. Panel (a) shows co-crystallization between CB[6] and (NH₄)₂PtCl₆. When a 4 M HCl solution of CB[6] (1.0 mL, 10 mM) was added to an aqueous solution of (NH₄)₂PtCl₆ (1 mL, 10 mM), a yellow suspension formed immediately. Panel (b) shows powder X-ray diffraction patterns of CB[6]·(NH₄)₂PtCl₆ and CB[6]·H₂PtCl₆ adducts, respectively.

FIG. 16. X-Ray photoelectron spectra of the CB[6]·H₂PtCl₆ microcrystals and the microcrystals obtained by adding CB[6] to an aqueous solution of [PtCl₆]²⁻, [PdCl₄]²⁻ and [RhCl₆]³⁻ anions, followed by washing with H₂O.

FIG. 17. Effect of changes in concentration of CB[6] and H₂PtCl₆ on Pt-precipitation yields based on the co-precipitation of CB[6]·H₂PtCl₆ adducts. The concentration of HCl was 2 M in all suspension solutions of CB[6]·H₂PtCl₆.

FIG. 18. Panel (a) shows capped-sticks representation showing how the [PtCl₆]²⁻ (1) interacts with six CB[6] (A-F) in the solid-state superstructure of CB[6]·H₂PtCl₆. Panel (b) shows results of DFT calculations of the binding energies between the [PtCl₆]²⁻ (1) and six adjacent CB[6] (A-F).

FIG. 19. Panels (a) and (b) show capped-sticks representations showing how the CB[6](A, F) interact with six adjacent [PtCl₆]²⁻ (1-6) in the solid-state superstructure of CB[6]·H₂PtCl₆, respectively. Panels (c) and (d) show results of DFT calculations of the binding energies between the CB[6] (A, F) and six adjacent [PtCl₆]²⁻ (1-6), respectively.

FIG. 20. Panel (a) shows capped-sticks representation showing how the [PtCl₄]²⁻ (1) interacts with five CB[6] (A-E) in the solid-state superstructure of CB[6]·H₂PtCl₄. Panel (b) shows results of DFT calculations of the binding energies between the [PtCl₄]²⁻ (1) and five adjacent CB[6] (A-E).

FIG. 21. Panels (a) through (e) show capped-sticks representations showing how the CB[6] (A-E) interact with their adjacent [PtCl₄]²⁻ in the solid-state superstructure of CB[6]·H₂PtCl₄, respectively. Panels (f) through (j) show results of DFT calculations of the binding energies between the CB[6] (A-E) and their adjacent [PtCl₄]²⁻, respectively.

FIG. 22. Panel (a) shows capped-sticks representation showing how the [PdCl₄]²⁻ (1) interacts with five CB[6] (A-E) in the solid-state superstructure of CB[6]·H₂PdCl₄. Panel (b) shows results of DFT calculations of the binding energies between the [PdCl₄]²⁻ (1) and five adjacent CB[6] (A-E).

FIG. 23. Panels (a) through (e) show capped-sticks representations showing how the CB[6] (A-E) interact with their adjacent [PdCl₄]²⁻ in the solid-state superstructure of CB[6]·H₂PdCl₄, respectively. Panels (f) through (j) Results of DFT calculations of the binding energies between the CB[6] (A-E) and their adjacent [PdCl₄]²⁻, respectively.

FIG. 24. Panel (a) shows capped-sticks representation showing how the [RhCl₆]³⁻ (1) interacts with six CB[6] (A-F) in the solid-state superstructure of CB[6]·H₃RhCl₆. Panel (b) shows results of DFT calculations of the binding energies between the [RhCl₆]³⁻ (1) and six adjacent CB[6] (A-F).

FIG. 25. Panels (a) through (f) show capped-sticks representations showing how the CB[6] (A-F) interact with two adjacent [RhCl₆]³⁻ (1-2) in the solid-state superstructure of CB[6]⁻ H₃RhCl₆, respectively. Panels (g) through (1) show results of DFT calculations of the binding energies between the CB[6] (A-F) and two adjacent [RhCl₆]³⁻ (1-2), respectively.

FIG. 26. Binding modes (above) and binding energies (below) of adjacent CB[6] molecules in the four single crystals.

FIG. 27. Color-coded sign (λ₂)_{ρ} scale bar.

FIG. 28. Panel (a) shows top-down and Panels (b) and (c) side-on views of the ball-and-stick representations of a CB[6] molecule interacting with six [PtCl₆]²⁻ dianions, as visualized by the intermolecular binding iso-surface. Ax inter (p)=0.003 a.u. Iso-surfaces are shaded over the range −0.05<sign(λ2)ρ<+0.05 a.u.

FIG. 29. Top-down (a) and bottom-up views (b) of the ball-and-stick representation of a [PtCl₆]²⁻ dianion interacting with six CB[6] molecules, as visualized by the intermolecular binding iso-surface. Ax inter (p)=0.003 a.u. Iso-surfaces are shaded over the range −0.05<sign(λ2)ρ<+0.05 a.u.

### DESCRIPTION OF THE INVENTION

Disclosed herein are high-efficiency platinum recovery methods utilizing cucurbiturial. The composition and methods described herein provide an environmentally benign, highly efficient, and thoroughly selective processes for platinum recovery. As further described herein, contacting a macrocycle with platinum halide anions creates adducts where the platinum halide anions are reversibly bound to the outer surface of the macrocycle by non-covalent interactions, allowing of the efficient production of precipitates that may be separated from their platinum bearing source material.

The Exampled demonstrate instantaneous co-crystallization and concomitant co-precipitation of dianions through noncovalent bonding interactions on the outer surface of a cucurbitil, such as [PtCl₆]²⁻ dianions and cucurbit[6]uril, a phenomenon which relies on the selective recognition of these dianions through noncovalent bonding interactions on the outer surface of cucurbit[6]uril. The selective [PtCl₆]²⁻ dianion recognition is driven by the weak [Pt—Cl . . . H—C] hydrogen bonding and [Pt—Cl . . . C═O] ion-dipole interactions. The synthetic protocol is highly selective. It is not observed in combinations between cucurbit[6]uril and other Pt- and Pd- or Rh-based chloride anions. We have also demonstrated that cucurbit[6]uril is able to separate selectively [PtCl₆]²⁻ dianions from a mixture of [PtCl₆]²⁻, [PdCl₄]²⁻ and [RhCl₆]³⁻ anions. This highly selective and fast co-crystallization protocol allows for recovery of platinum from spent vehicular three-way catalytic converters and other platinum-bearing metal waste.

An “adduct” is a new chemical species AB, each molecular entity of which is formed by direct combination of two separate molecular entities A and B in such a way that there is change in connectivity, but no loss, of atoms within the moieties A and B. Stoichiometries other than 1:1 are also possible, such as 2:1, 3:1, 4:1 and so forth.

The adduct is formed from a metal halide anion non-covalently bound to the outer surface of a macrocycle. Macrocycles are a cyclic macromolecular or a macromolecular cyclic portion of a macromolecule. A molecule of high relative molecular mass, the structure of which essentially comprises the multiple repetition of units derived, actually or conceptually, from molecules of low relative molecular mass.

Suitably the macrocycle is a cucurbituril. A “cucurbituril” is macrocyclic molecule made of glycoluril (═C₄H₂N₄O₂═) monomers linked by methylene bridges (—CH₂—). Cucurbituril may generically be referred to as cucurbit[n]uril or CB[n] wherein n is the number of glycoluril units. An exemplary method of preparing CB[n] is shown in Scheme 1.

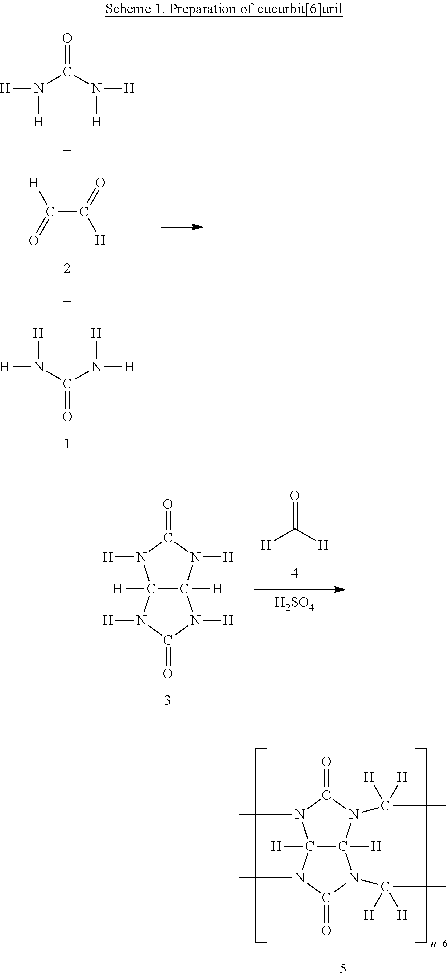

Cucurbiturils are amidals and synthesized from urea 1 and a dialdehyde (e.g., glyoxal 2) via a nucleophilic addition to give the intermediate glycoluril 3. This intermediate is condensed with formaldehyde to give hexamer cucurbit[6]uril above 110° C. Ordinarily, multifunctional monomers such as 3 would undergo a step-growth polymerization that would give a distribution of products, but due to favorable strain and an abundance of hydrogen bonding, the hexamer 5 is the only reaction product isolated after precipitation.

Other cucurbiturils may also be prepared with differing numbers of glycoluril units. Decreasing the temperature of the reaction to allows access to other sizes of cucurbiturils, including CB[5], CB[7], CB[8], and CB[10]. CB[6] may still be the major product with the other ring sizes formed in smaller yields. The isolation of sizes other than CB[6] requires fractional crystallization and dissolution.

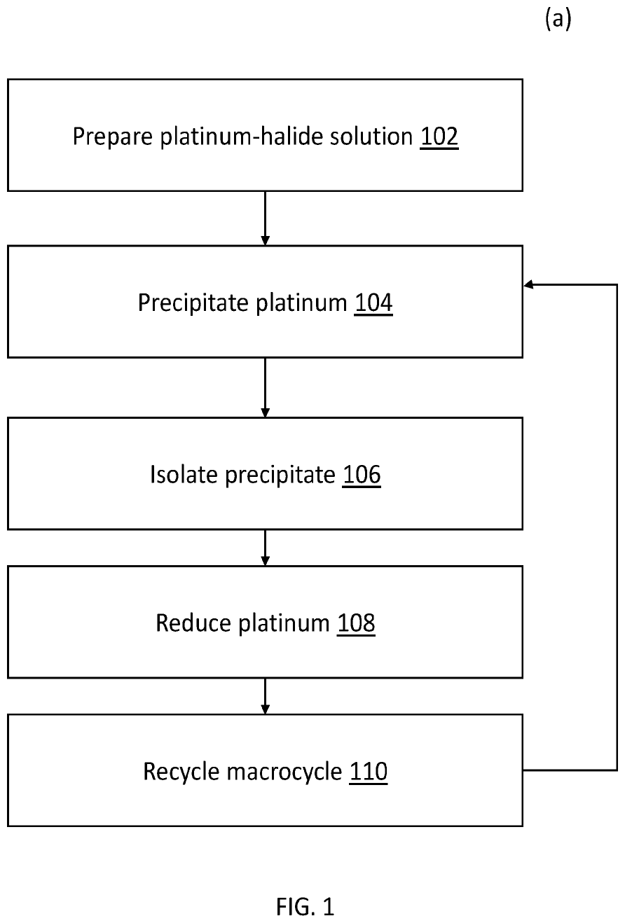

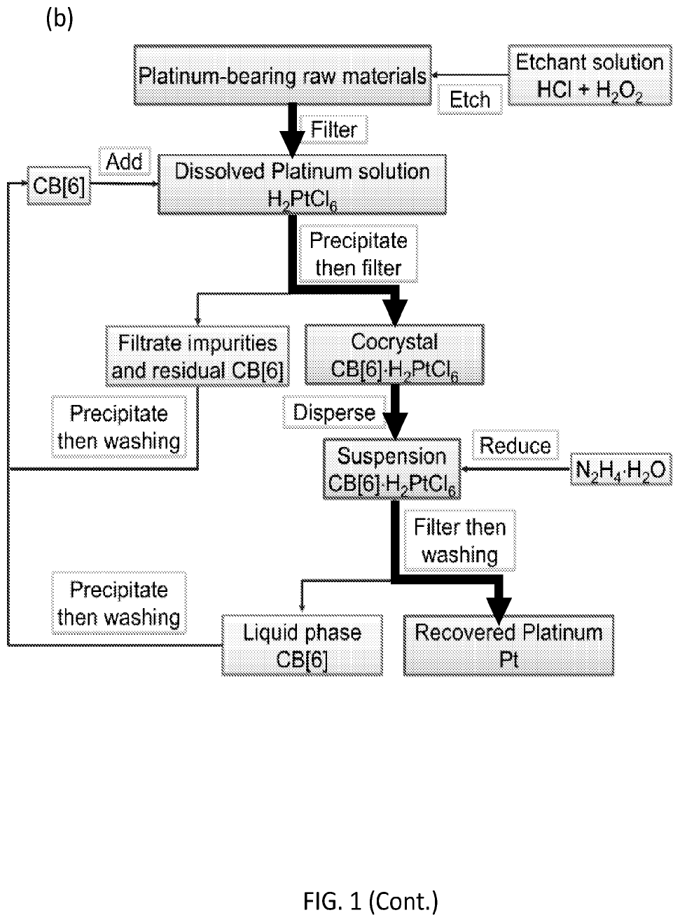

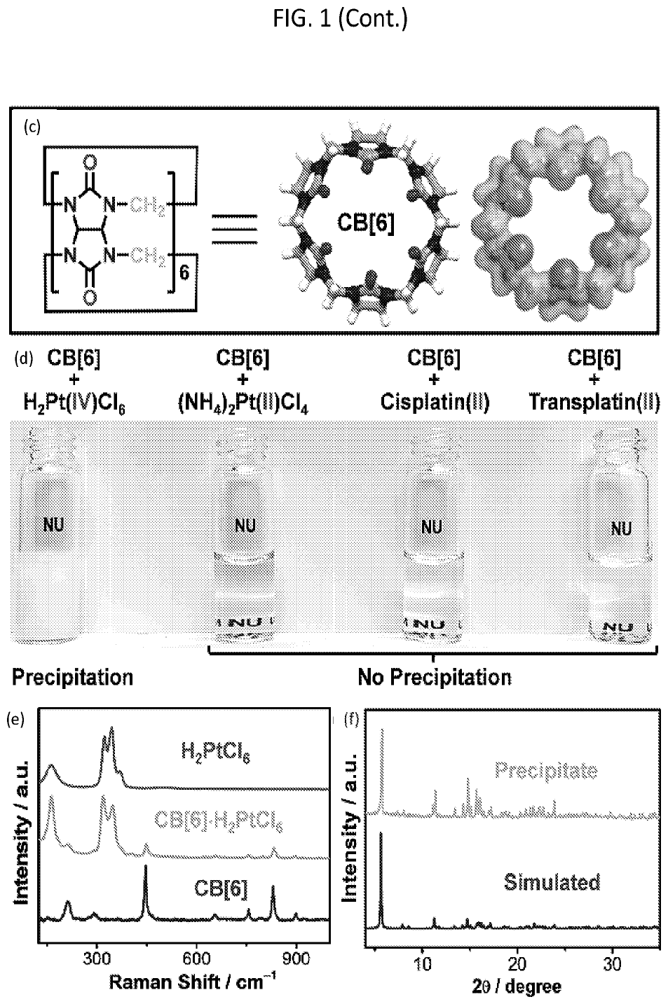

Cucurbit[6]uril (CB[6]) possesses (FIG. 1 (c)), partially negatively charged, carbonyl-fringed portals, while the C—H bonds on its outer surface are slightly electrostatically positive. On account of the electron-rich nature of its carbonyl-fringed portals and well-defined hydrophobic cavity, the binding behavior by CB[6] toward various metal cations, as well as organic ammonium and imidazolium cations, has been investigated. By contrast, reports relating to their exo-binding properties, based on the outer surface interactions with CB[6], are still rather limited. Selective anion recognition and separation employing outer surface interactions with cucurbiturils has been little investigated to date.

The metal halide anion comprises a noble metal. In some embodiments, the metal halide anion is a octahedral anion such as [MX₆]²⁻ where M is a noble metal, such as Pt, and X is a halide. Each halide may be the same, such as for [PtCl₆]²⁻ or [PtBr₆]²⁻. In other embodiments, the metal halide anion may comprise two or more different halides. In some embodiments, the metal halide anion is provided with a counter anion such as H⁺ or metal cation, such as an alkali cation.

Superstructures and crystalline compositions may be formed from adducts described herein. A “crystalline composition” is a material whose constituents are arranged in a highly ordered microscopic structure, forming a crystal lattice. A “superstructure” is a material having additional structure superimposed upon a given crystalline material, supramolecular assembly, or other well-defined substructure.

A “supramolecular assembly” is a well-defined complex of molecules held together by noncovalent bonds. Suitably, supramolecular assemblies may have well defined order in one, two, or three dimensions. The supramolecular assemblies described herein between the platinum halide anion and the outer surface of the macrocycle may include hydrogen bonds and/or ion-dipole interactions, such as [Pt—X . . . H—C] and [Pt—X . . . C═O], respectively.

The compositions described herein may be used for the isolation and recovery of Pt from platinum-bearing materials. A “platinum-bearing material” is material comprised of platinum atoms, regardless of oxidation state. Exemplary platinum-bearing materials include, without limitation, ores, metal mixtures, or post-consumer products.

The term “metal mixture” refers to two or more elements from Groups IA, IIA, IB to VIIIB, the lanthanide series and actinide series of the periodic table. An example of a metal mixture is Au and Pt.

The term “post-consumer product” refers to any man-made product for consumption, bartering, exchange or trade. Examples of “post-consumer product” include a catalytic material, jewelry item, an electronics item, precious metal products, and coins, among others.

The term “catalytic material” includes any substance that increases the rate of a chemical reaction without modifying the overall standard Gibbs energy change in the reaction. Catalytic materials may be homogeneous or heterogeneous. Exemplary catalytic materials include, without limitation, those found in a catalytic converter.

The term “jewelry item” includes any aesthetic item that includes as one component a precious metal. Examples of a jewelry item include a ring, a bracelet and a necklace, among others.

The term “electronics item” refers to a product that includes at least one circuit for conducting electron flow. Examples of an electronics item include a computer, a monitor, a power supply, an amplifier, and a preamplifier, a digital to analog converter, an analog to digital converter, and a phone, among others.

The term “precious metal product” includes a partially purified form or a purified form of a noble metal, such as gold, platinum, palladium and silver. Examples of a precious metal include a powder, ingot, or bar of gold, silver, platinum, among others. As used herein, “partially-purified form” refers to a form having from about 10% to about 75% of the pure form of a noble metal. As used herein, “purified form” refers to a form having greater than about 75% of the pure form of a noble metal.

The term “coin” refers to any pressed object composed of a pure metal, mixed metal or metal alloy that can be used as a currency, a collectable, among other uses. As used herein, “pure metal” refers to a single metal of at least 95% or greater purity. As used herein “mixed metal” refers to two or more metals. As used herein “metal alloy” refers to a mixture or solid solution of a metal with at least one other element.

A method for isolating and recovering platinum from platinum-bearing materials was developed based upon the selective co-precipitation of metal halide anions non-covalently bound to the outer surface of macrocycles. Referring to FIG. 1 (a), a platinum-bearing material is combined with a hydrogen halide (“HX”) and, optionally, an acid or H₂O₂ to form a platinum halide solution 102. Hydrogen halide can be any compound having the formula HX, wherein X is a halogen such as chlorine or bromine. The optional acid can include any strong acid, such as any of the foregoing hydrogen halides, or additionally HNO₃, H₂SO₄, among others. The pH of the platinum halide solution may be less than 4.0 or, in some embodiments, less than 3.0, or less than 2.0. The platinum of the platinum-bearing material reacts with the hydrogen halide to form the product HAuX₄.

The macrocycle may be added to the platinum halide solution to form a precipitate 104. Suitably the macrocycle is a cucurbituril, such as CB[6]. The precipitate may comprise any of the adducts, superstructure, or crystalline materials described herein. In some embodiments, the precipitate [PtX₆]²⁻ is bound to the outer surface of CB[6].

The precipitate is isolated from the metal halide salutation 106. Any means of isolation can be used to obtain precipitate, include filtration, centrifugation, and other separation methods known in the art.

In some embodiments, not all of platinum-bearing material can be dissolved in platinum halide solution. As a result, some solid remnants of platinum-bearing material (whether or not including platinum) can persist. In such aspects, it may be desirable to include a filtration step to remove the solid remnants prior to subsequent processing. The resultant filtrate may be processed as described above to obtain the isolated platinum.

The precipitate can be treated with a reducing agent to produce elemental platinum (Pt(0)) 108. Examples of reducing agents include, but are not limited to, N₂H₄, NaBH4, Na₂S₂O₅, and H₂C₂O₄, among others. The elemental platinum can be readily isolated as a precipitate and the macrocycle can be harvested in the liquid phase and recycled for reuse to precipitate additional platinum 110.

An exemplary aspect of the process outlined generally in FIG. 1 (a) is depicted in FIG. 1 (b).

The method for isolating and recovering platinum from platinum-bearing materials has several applications. In one aspect, the method can be applied to isolating platinum from platinum-bearing material, wherein the platinum-bearing material is selected from an ore, a metal mixture, or a post-consumer product. The foregoing examples of isolating platinum from platinum-bearing materials are not limited to the foregoing materials. The specific etching and leaching process for dissolving platinum from platinum-bearing materials results in formation of a specific platinum-halide compound that can be recovered in the form of a complex with the macrocycle, thereby rendering the method suitable for recovering platinum from each of these particular applications as well as other platinum-bearing materials.

The Examples demonstrate highly specific formation of a supramolecular crystalline material, facilitated by the selective recognition of [PtCl₆]²⁻ dianions on account of the outer surface interactions with CB[6]. The formation of the supramolecular cocrystals have been confirmed by Raman, X-ray photoelectron, SEM-equipped energy-dispersive X-ray spectroscopies, and their solid-state superstructures have been characterized by single-crystal and powder X-ray diffraction analyses. The rapid co-crystallization and spontaneous co-precipitation between CB[6] and [PtCl₆]²⁻, which is highly specific, do not manifest themselves in the case of six other Pt-, Pd-, and Rh-based chlorides. In proof-of-principle investigations, we have demonstrated the CB[6] can serve as an extractant to separate the [PtCl₆]²⁻ dianions in the presence of [PdCl₄]²⁻ and [RhCl₆]³⁻ anions. This highly selective and fast co-crystallization process may be used for platinum-recovery from spent three-way catalytic converters of vehicles.

Upon addition of a 4 M HCl solution of CB[6] (1.0 mL, 10.0 mM) to aqueous solutions of H₂PtCl₆, (NH₄)₂PtCl₄, cisplatin and transplatin (1.0 mL, 10.0 mM) at room temperature, a yellow suspension formed exclusively between H₂PtCl₆ and CB[6]. See FIG. 1 (d). This observation establishes the fact that CB[6] is able to recognize selectively the Pt(IV) chloride anion, rather than Pt(II) chloride. Filtration and air drying of the co-precipitate permits isolation of a pale yellow solid. The Raman spectrum of this yellow solid shows (FIG. 1 (e)) sharp vibrational bands for H₂PtCl₆ at 163, 320, and 348 cm⁻¹, in addition to the characteristic vibration bands, accompanied with broadening, for CB[6] at 448 and 833 cm⁻¹. These data confirm the formation of an adduct between CB[6] and H₂PtCl₆, namely, CB[6]·H₂PtCl₆. In order to investigate the crystallinity of the CB[6]·H₂PtCl₆ adduct, powder X-ray diffraction (PXRD) analysis was performed. The PXRD pattern shows (FIG. 1 (f)) a series of sharp diffraction peaks, indicating that the co-precipitate is a highly crystalline material. This observation demonstrates that we can obtain almost instantaneously an organic-inorganic hybrid supramolecular cocrystal as a result of the rapid crystallization between CB[6] and H₂PtCl₆.

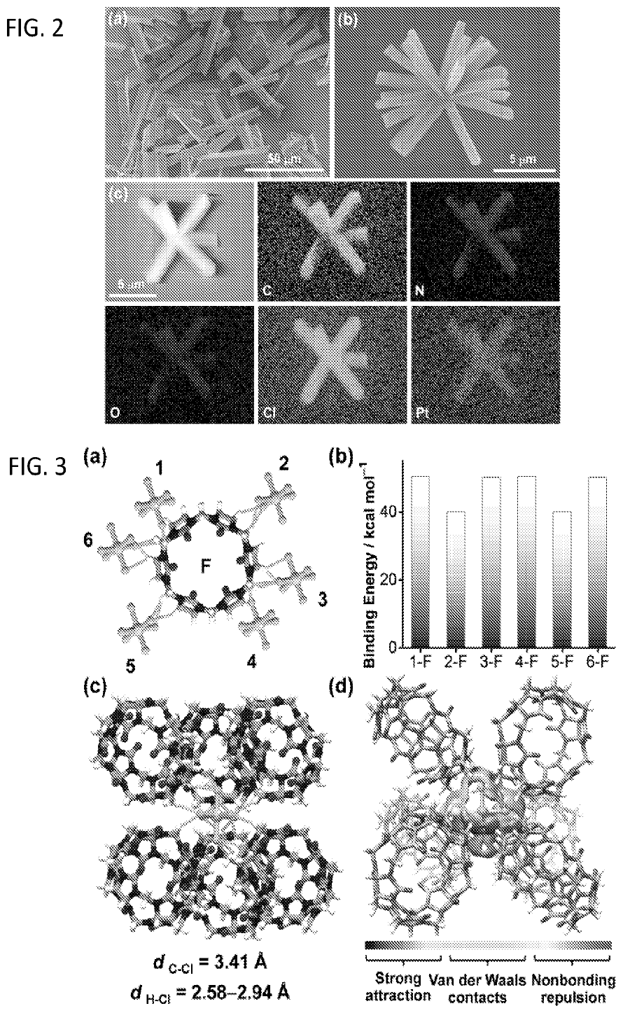

Scanning electron microscopy (SEM) was employed to investigate the topological morphology and elemental distribution in these microcrystals formed between CB[6] and H₂PtCl₆. In the SEM images (FIG. 2) of the air-dried aqueous suspension of CB[6]·H₂PtCl₆, a plethora of angular rod-like microstructures with diameters in the range of several micrometers and with lengths up to a few dozens of micrometers were observed. Some of the CB[6]·H₂PtCl₆ microcrystals were hollow (FIGS. 2 (a) and 11). The small microrods are inclined to form (FIG. 2 (b)) flower-like aggregates. SEM-Equipped energy-dispersive X-ray spectroscopic (SEM-EDS) elemental maps reveal (FIG. 2 (c)) a homogeneous distribution of the elements of carbon, nitrogen, oxygen, chlorine, and platinum throughout the microrods, confirming the formation of CB[6]·H₂PtCl₆ adducts.

In order to gain insight into the noncovalent bonding forces behind the instantaneous co-crystallization between CB[6] and H₂PtCl₆, a single-crystal X-ray diffraction analysis was carried out. High quality yellow crystals of CB[6]·H₂PtCl₆ were obtained by the slow diffusion of an aqueous H₂PtCl₆ solution into a 4 M HCl solution of CB[6] during the course of 6 h. CB[6]·H₂PtCl₆ Crystallizes (FIG. 3 and Table 2) in the monoclinic space group I2/a, which is the same in terms of crystal morphology as that obtained by evaporating slowly a mixture solution of CB[6] and K₂PtCl₆ in 6 M HCl aqueous solution during the course of 6 days as described in a previous report^{[39]}. These results indicate the K⁺ metal ions do not influence the selective co-crystallization between CB[6] and [PtCl₆]²⁻ even during a slow solvent evaporation process.

In the solid-state superstructure, the stoichiometry between CB[6] and [PtCl₆]²⁻ is 1:1, and [PtCl₆]²⁻ dianions interact (FIG. 3) with the outer surface of CB[6], rather than reside inside the cavities of the cucurbiturils. Each CB[6] molecules interacts (FIG. 3 (a)) with six [PtCl₆]²⁻ dianions by means of [Pt—Cl H—C] hydrogen bonds and [Pt—Cl . . . C═O] ion-dipole interactions with the distances of 2.6-2.9 and 3.4 Å, respectively. Density functional theory (DFT) calculations revealed (FIG. 3 (b)) that the binding energy between CB[6] and [PtCl₆]²⁻ ranges from 40.0 to 50.5 kcal mol⁻¹, values which are larger than those recorded^{[35d]} between CB[6] and [AuCl₄]⁻. In turn, every [PtCl₆]²⁻ dianion is surrounded (FIG. 3 (c)) by six CB[6] molecules, and stabilized by 14 sets of hydrogen bonds as well as two sets of ion-dipole interactions. Independent gradient model (IGM) analysis provided (FIG. 3 (d)) the visualized information for these noncovalent bonding interactions. It revealed that, except for the hydrogen bonding and ion-dipole interactions, van der Waals interactions are also involved in sustaining this binding mode (FIG. 3 (c)). The simulated PXRD pattern, based on the single-crystal X-ray data of CB[6]·H₂PtCl₆, matches (FIG. 1 (f)) well with the experimental one, indicating that the superstructure of the CB[6]·H₂PtCl₆ microcrystals obtained by solution-phase synthesis are consistent with its single-crystal X-ray diffraction analysis.

We also mixed CB[6] with other platinum group metal anions, e.g., [PdCl₄]²⁻ dianions with square-planar geometry, [PdCl₆]²⁻ and [RhCl₆]³⁻ anions with regular-octahedral geometries. None of these three combinations—i.e., CB[6]⁻ [PdCl₄]²⁻, CB[6]⁻ [PdCl₆]²⁻, and CB[6]⁻ [RhCl₆]³ produced a precipitate (FIG. 6). In order to gain further insight into the different crystallization behavior and binding energies of CB[6] and these anions, single-crystal X-ray diffraction analyses and DFT calculations were carried out. Single crystals^{[40]} of all three adducts, i.e., CB[6]⁻ [PtCl₄]²⁻, CB[6]·[PdCl₄]²⁻ and CB[6]⁻ [RhCl₆]³⁻, were grown during the course of one week by evaporating slowly aqueous solutions containing 1:1 mixtures of CB[6] and either (NH₄)₂PtCl₄, (NH₄)₂PdCl₄, or (NH₄)₃RhCl₆.

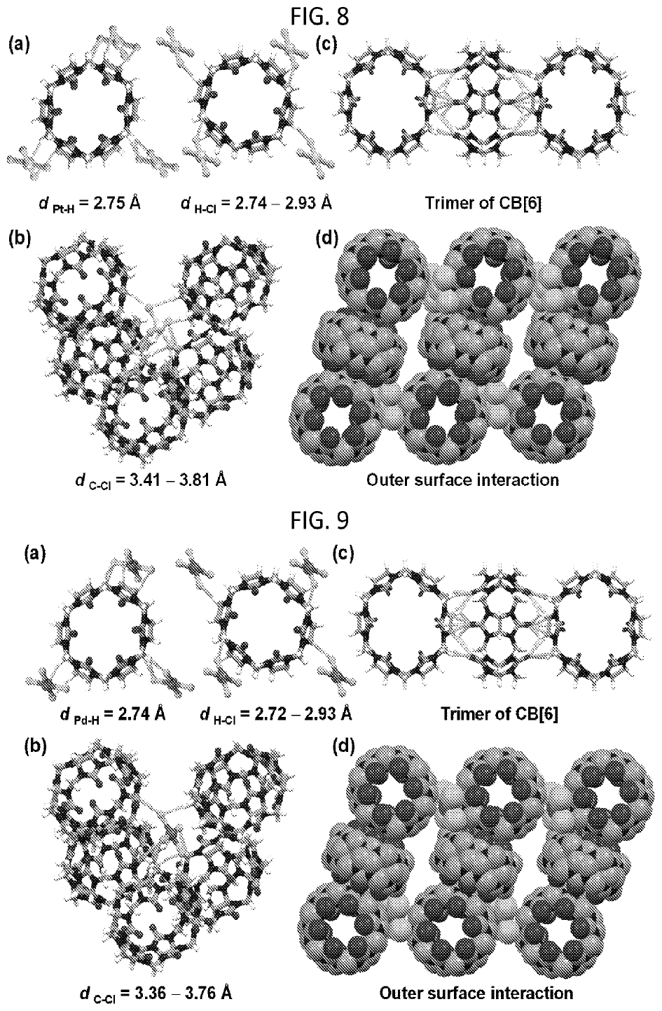

Single-crystal X-ray diffraction analyses reveal (Table 2) that NH₄ ions are absent in all the crystal structures. A possible explanation is that NH₄ ions are replaced by protons in aqueous acidic solution during crystallization. This observation indicates that anions play a crucial role in the crystallization, while the effect of the NH₄ ions is negligible. All the [PtCl₄]²⁻, [PdCl₄]²⁻, and [RhCl₆]³⁻ anions co-crystallize with CB[6], sustained (FIGS. 8-10) principally by multiple hydrogen bonds and ion-dipole interactions, similar to those (FIG. 3) involving the [PtCl₆]²⁻ dianions.

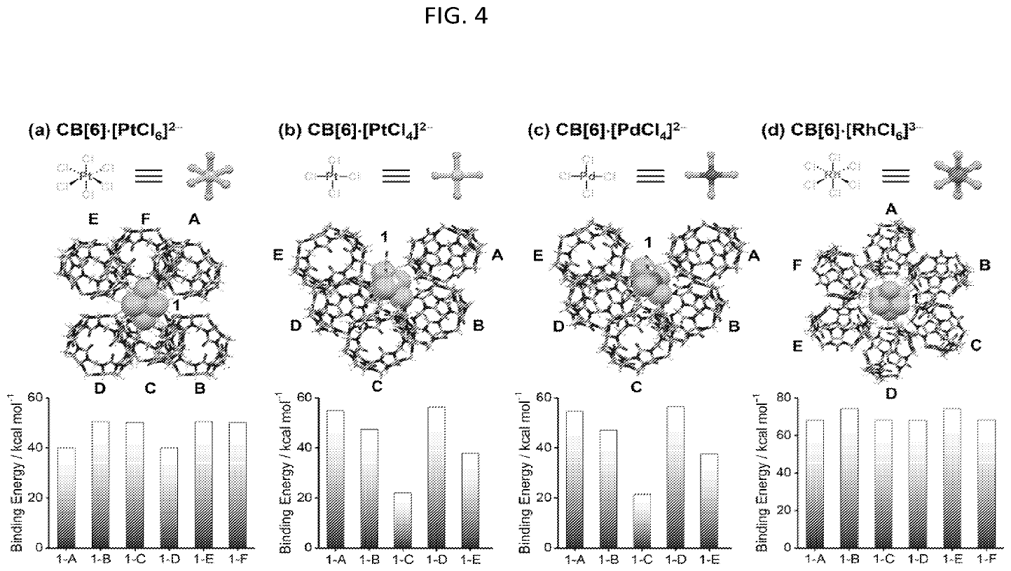

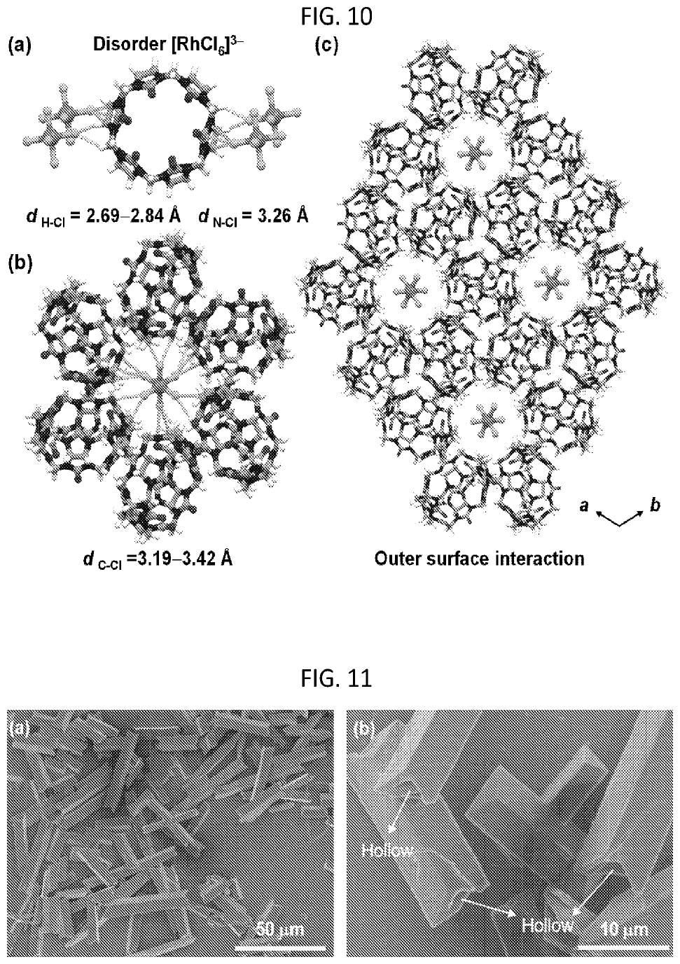

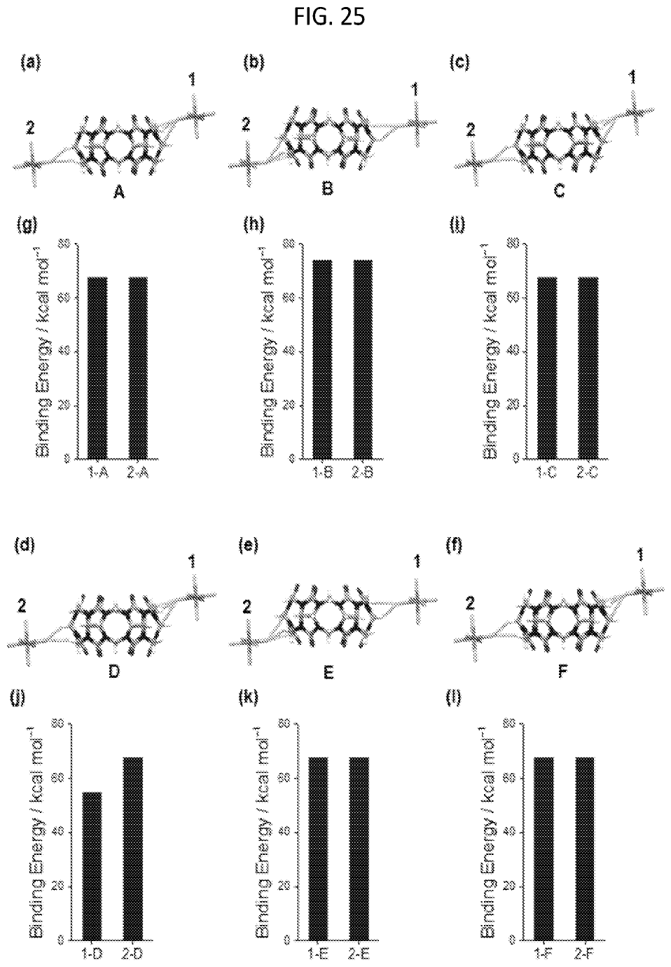

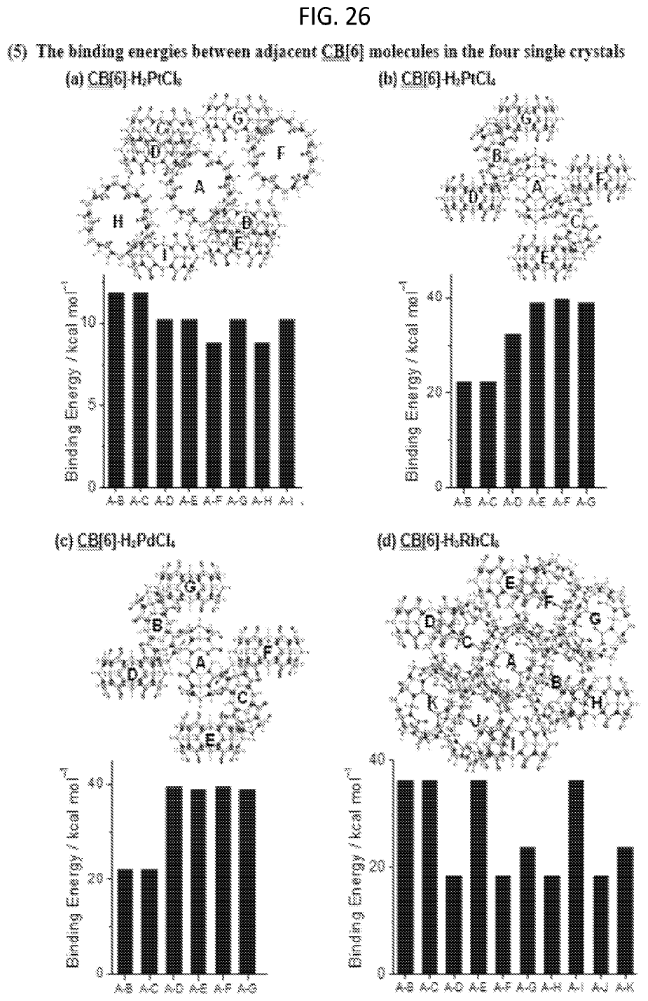

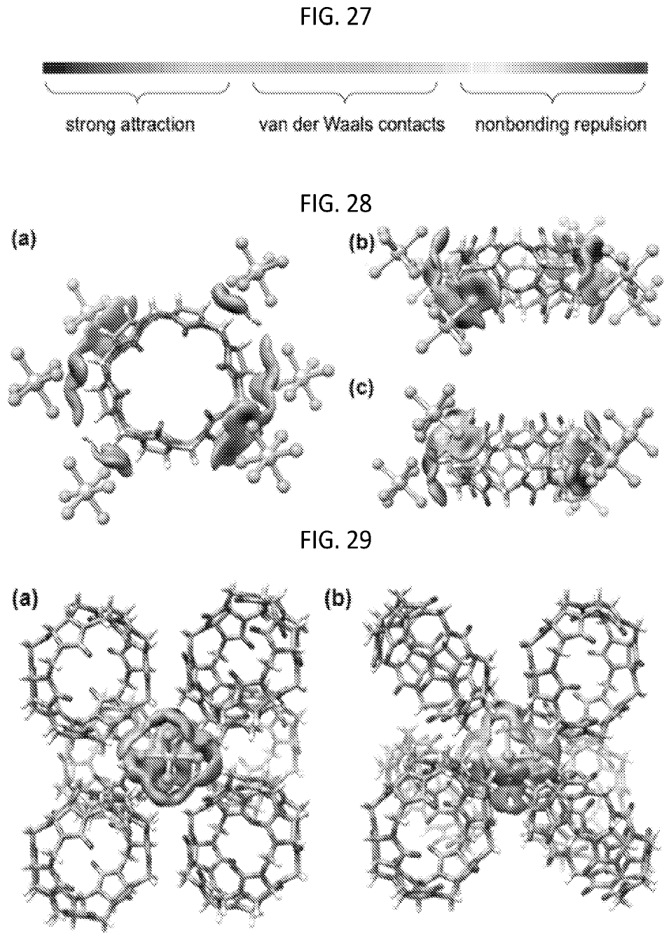

The specific assembly and binding modes between CB[6] and the four anions are different. In the solid-state superstructures, CB[6]·[PtCl₄]²⁻ and CB[6]⁻ [PdCl₄]²⁻ adopt (Table 2) the same space group, P2₁/c. The [MCl₄]²⁻ (M=Pt/Pd) dianions reside in (FIGS. 8-9) the lattice space between the parallelly arranged trimers of CB[6] molecules. Whereas every [MCl₄]²⁻ (M=Pt/Pd) dianion interacts (FIG. 4 (b)-(c)) with five CB[6] and several H₂O molecules, by contrast, the [PtCl₆]²⁻ dianion is only trapped by six CB[6] molecules. The additional coordination of H₂O molecules on the [MCl₄]²⁻ (M=Pt/Pd) dianions, not only keeps these adducts solvated, but also competes with the coordination of CB[6] during the assembly process, hence restricting precipitation. DFT Calculations reveal (FIG. 4) that each CB[6] molecule has a different binding energy toward the central anion on account of the fact that they possess different bonding interactions and distances relative to the central anion. The total binding energies (Table 9) between the central [PtCl₄]²⁻ and [PdCl₄]²⁻ dianions and their surrounding five CB[6] molecules are 113.0 and 115.4 kcal mol⁻¹, respectively, values which are both lower than the binding energy (136.4 kcal mol⁻¹) between the [PtCl₆]²⁻ dianion and its surrounding six CB[6] molecules. In the case of the CB[6]⁻ [RhCl₆]³⁻ adduct, the [RhCl₆]³⁻ trianions reside (FIG. 10 (c)) in the channels of a honeycomb-like organic framework formed by CB[6] molecules. Every [RhCl₆]³⁻ trianion is disordered (FIG. 10 (a)) over two positions with 50:50 occupancies, indicating the potential motility of the [RhCl₆]³⁻ trianions in these channels. In the solid-state superstructure, each CB[6] molecule is attached (FIG. 25) to two [RhCl₆]³⁻ trianions, whereas up to six [PtCl₆]²⁻ dianions are bound (FIG. 3 (a)) to one CB[6] molecule in the cocrystals of CB[6]·H₂PtCl₆. Every [RhCl₆]³ trianion interacts (FIG. 4 (d)) with six CB[6] molecules, a situation which is similar to that involving the [PtCl₆]²⁻ dianion. The total binding energy (Table 9) between the central [RhCl₆]³⁻ trianion and its surrounding six CB[6] molecules is 226.7 kcal mol⁻¹. One possible reason for the lack of precipitation in the case of CB[6] and [RhCl₆]³⁻ is that neighboring CB[6]⁻ [RhCl₆]³⁻ clusters possess stronger electrostatic repulsion compared to that for the CB[6]⁻ [PtCl₆]²⁻ clusters. The binding energy between adjacent CB[6] molecules in the four crystals was also investigated by DFT calculations. In the case of the CB[6]⁻ [PtCl₆]²⁻ adduct, the binding energy (FIG. 26 (a)) between two neighboring CB[6] molecules ranges from 8.9 to 11.9 kcal mol⁻¹, values which are much lower than those (18.7-39.9 kcal mol⁻¹) obtained for the other three adducts (FIG. 26 (b)-(d)).

The single-crystal X-ray diffraction analyses and DFT calculations performed on the four adducts formed between CB[6] molecules and the [PtCl₆]²⁻, [PtCl₄]²⁻, [PdCl₄]²⁻, [RhCl₆]³⁻ anions led us to deduce possible factors for the promotion of rapid co-crystallization between CB[6] and the anions, including (i) highly specific intermolecular hydrogen bonds and ion-dipole interactions between the outer surface of CB[6] and the anions, which dominate the driving forces for forming the macrocycle-anion adducts and long-range ordered crystals, (ii) weak electrostatic repulsions between CB[6]⁻ anion clusters, which favor the formation of bulk crystalline assemblies, and (iii) the low degrees of solvation of the resulting assemblies formed by these clusters, accelerating their precipitation from solution.

This selective co-crystallization between CB[6] and H₂PtCl₆ motivated us to test the validity of employing CB[6] for platinum separation. In an attempt to obtain an estimate of the co-precipitation efficiency, the suspension of the CB[6]·H₂PtCl₆ adduct was filtered, and the filtrate was subjected to inductively coupled plasma optical emission spectroscopic (ICP-OES) analysis in order to determine the concentration of [PtCl₆]²⁻ dianions remaining. On the basis of the initial and residual concentrations of these dianions in the aqueous solution, the yield of precipitated [PtCl₆]²⁻ can be obtained. The platinum-precipitation yield, based on the cocrystals of CB[6]·H₂PtCl₆, (FIG. 17 and Table 3) is 80.1% at a concentration of 5.0 mM. When the concentration of the CB[6]·H₂PtCl₆ adduct is increased from 2 to 10 mM, the platinum-precipitation yield ranges (FIG. 17 and Table 3) from 73.9 to 80.7%. These results indicate that the instantaneous co-crystallization between CB[6] and H₂PtCl₆ is retained over a wide range of concentrations.

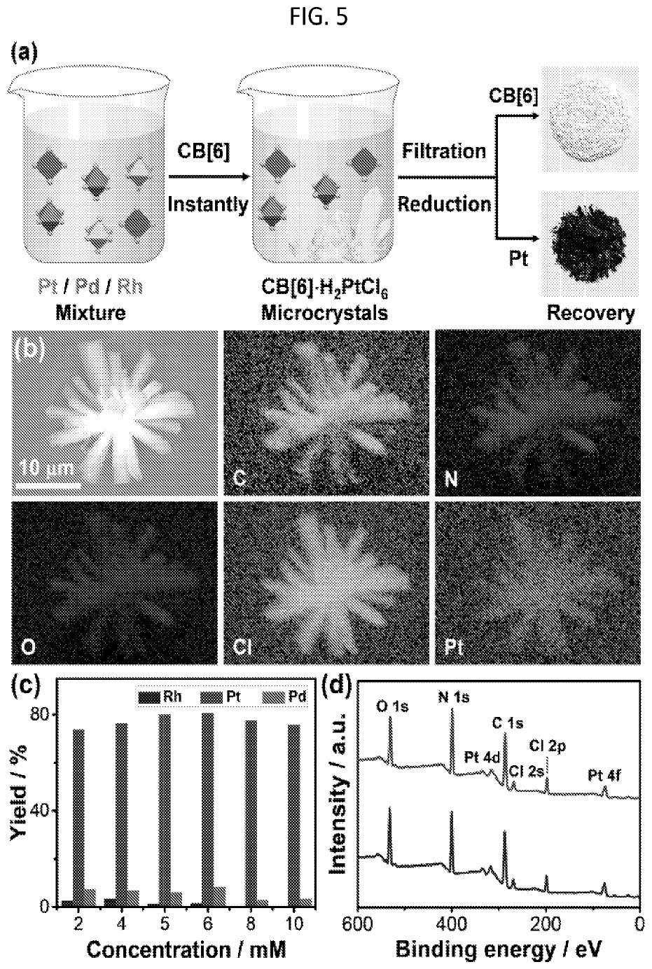

Three-way catalytic converters of vehicles,^{[41]} containing considerable amounts of precious platinum, palladium, and rhodium metals, are vital components for converting the harmful nitrogen oxides (NOx), hydrocarbons, and carbon monoxide (CO) emitted from engines into relatively harmless nitrogen (N₂) and carbon dioxide (CO₂). With many vehicles reaching the end of their useful lives, a large number of waste catalytic converters are produced every year. From an economic as well as an environmental perspective, the recovery^{[42]} of platinum from spent catalytic converters is vital. In order to test the potential of employing CB[6] as an extractant to separate platinum from spent three-way catalytic converters, we attempted to precipitate (FIG. 5 (a)) platinum as its [PtCl₆]²⁻ dianion from an aqueous solution containing equimolar amounts of [PtCl₆]²⁻, [PdCl₄]²⁻ and [RhCl₆]³⁻ anions. When an aqueous solution of CB[6] was added to this mixture, yellow microcrystals formed immediately.

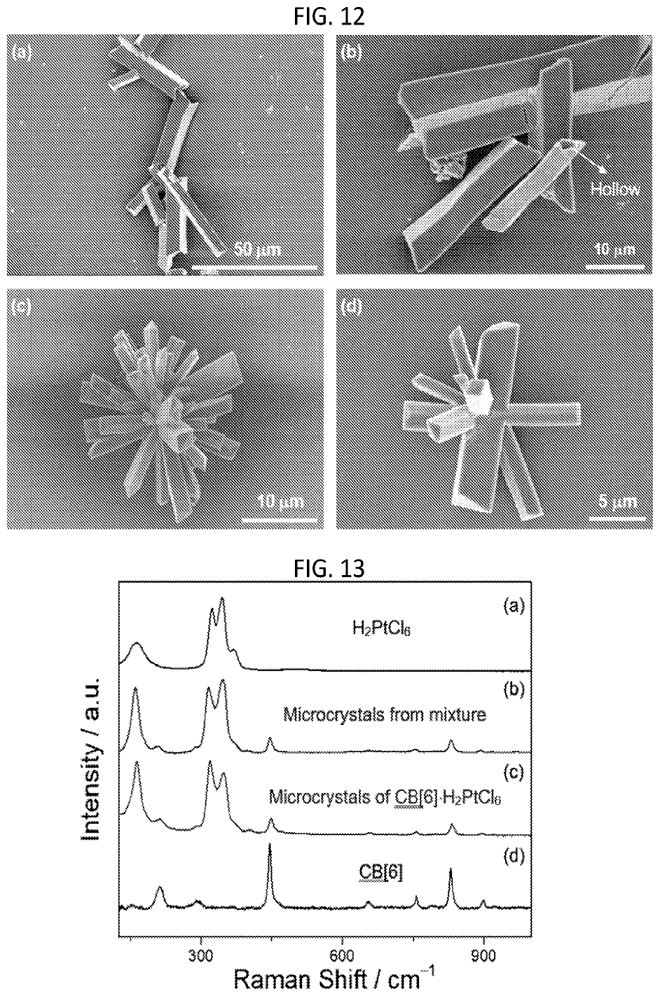

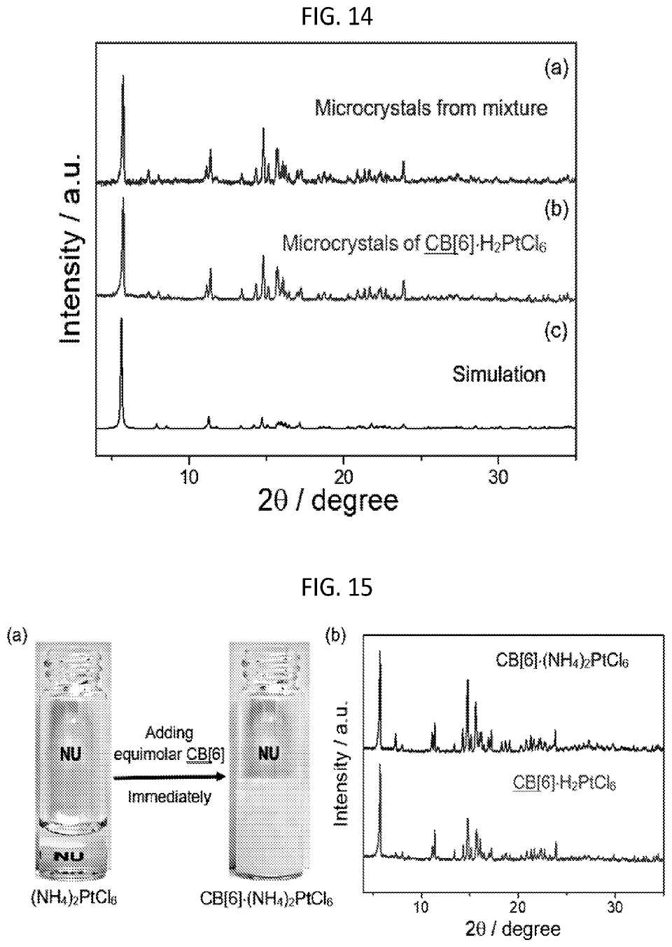

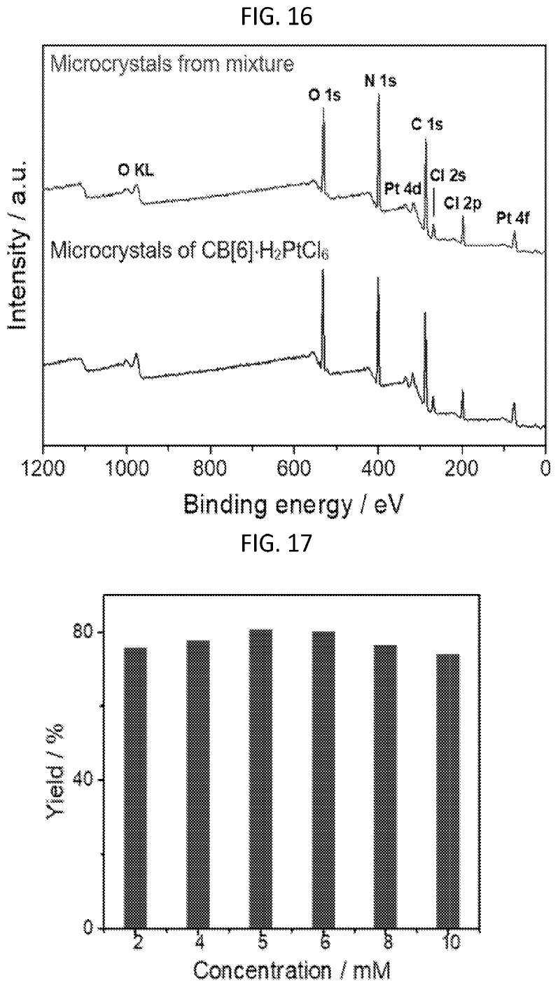

The Raman (FIG. 13) and PXRD (FIG. 14) spectra of these microcrystals match well with the isolated CB[6]·H₂PtCl₆ adduct. These observations indicate that the presence of [PdCl₄]²⁻ and [RhCl₆]³⁻ anions has a negligible impact on the co-crystallization between CB[6] and [PtCl₆]²⁻. SEM Analysis revealed that the microstructure of the yellow microcrystals is similar to that of CB[6]·H₂PtCl₆ adduct. Plenty of solid and hollow microrods, and flower-like aggregates, were observed (FIG. 12) in the SEM images. SEM-EDS Revealed (FIG. 5 (b)) that all the elements, including carbon, nitrogen, oxygen, chlorine, and platinum, were distributed uniformly in the microrods. The ICP-OES analysis (FIG. 5 (c)) indicated 79.3% of [PtCl₆]²⁻ dianions in the original mixture separates out from the solution at a concentration of 5.0 mM, a value which is close to the precipitation efficiency (80.1%) obtained when adding CB[6] to a solution of only [PtCl₆]²⁻ dianions. Since 6.1% of Pd and 1.3% of Rh were also removed from the mixture at a concentration of 5.0 mM according to the ICP-OES analyses (FIG. 5 (c)), we suspect that small amounts of [PdCl₄]²⁻ and [RhCl₆]³ anions may have remained in the hollow microcrystals of CB[6]·H₂PtCl₆. We attempted to remove the residual [PdCl₄]²⁻ and [RhCl₆]³⁻ anions by washing the CB[6]·H₂PtCl₆ microcrystals with H₂O. In the X-ray photoelectron spectra (XPS) of the washed microcrystals, only the characteristic peaks of carbon, nitrogen, oxygen, chlorine, platinum were observed (FIG. 5 (d)), whereas signals for palladium and rhodium were absent. These results demonstrate that CB[6] molecules are highly selective for forming cocrystals with [PtCl₆]²⁻ dianions, even in the presence of [PdCl₄]²⁻ and [RhCl₆]³⁻ anions, and that CB[6]·H₂PtCl₆ microcrystals can be isolated by straightforward filtration and washing.

In an attempt to recover the platinum metal from the CB[6]·H₂PtCl₆ microcrystals, the adducts were dispersed in an aqueous solution, and adding N₂H₄·H₂O, so as to reduce the [PtCl₆]²⁻ dianions down to platinum. After centrifuging and washing with aqueous acid solution to dissolve the residual CB[6], black platinum metal (FIG. 5 (a)) was isolated. About 89.3% of platinum was recovered (Table 1) from the microcrystals. ICP-OES Analysis revealed (Table 8) the purity of the platinum metal is 94.9%. In addition, CB[6] molecules, which dissolves in the solution, can be (FIG. 5 (a)) recycled by precipitating with acetone, and can be used (Table 1) during subsequent rounds of platinum recovery after washing. Based on the selective co-crystallization between CB[6] and H₂PtCl₆, a platinum-recovery flow diagram (FIG. 1 (b)) has been proposed.

The process provide an alternative supramolecular based protocol for the recovery of platinum from platinum-bearing scrap.

We have shown that the instantaneous co-crystallization of CB[6] and H₂PtCl₆, which leads to rapid co-precipitation of the CB[6]·H₂PtCl₆ adduct, is sustained by multiple weak [Pt—Cl . . . H—C] hydrogen bonds and [Pt—Cl . . . C═O] ion-dipole interactions. This rapid co-crystallization and concomitant co-precipitation only occurs between CB[6] and H₂PtCl₆, despite the fact that similar outer surface interactions between CB[6] and other platinum group metal chloride anions are present. The selective recognition of [PtCl₆]²⁻ dianions through outer surface interactions with CB[6], not only expands the scope of the second-sphere coordination^{[29b, 32, 37c]} of metal complexes on the part of cucurbituril, but also casts more light on the outer surface interactions by macrocycles. Additionally, this rapid co-precipitation between CB[6] molecules and [PtCl₆]²⁻ dianions can be applied to the extraction of platinum from aqueous solution. We have also demonstrated that CB[6] is able to separate selectively [PtCl₆]²⁻ dianions in the presence of [PdCl₄]²⁻ and [RhCl₆]³⁻ anions, and that the platinum metal can be recovered easily after reducing the co-precipitate with N₂H₄·H₂O. This technology allows for the recovery platinum from the spent vehicular three-way catalytic converters as well as other platinum-bearing metal waste.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Materials and Instrumentation

### (1) Materials

All the chlorides, including H₂PtCl₆, (NH₄)₂PtCl₆, (NH₄)₂PtCl₄, cisplatin, transplatin, (NH₄)₂PdCl₆, (NH₄)₂PdCl₄, (NH₄)₃RhCl₆, HCl (wt 37%) and N₂H₄·H₂O (wt 78%) aqueous solutions were purchased from commercial suppliers and used without further purification unless stated otherwise. The cucurbit[6]uril was synthesized according to the previous literature with some modifications¹. High purity water was generated by a Milli-Q.

### (2) Scanning Electron Microscopy

Scanning electron microscopy (SEM) images were performed on a SU8030 scanning electron microscope at the voltages of 5.0/10 kV, while the energy-dispersive X-ray spectroscopy (EDS) elemental maps were recorded at 40 kV.

### (3) Raman Spectroscopy

Raman spectra were performed on a HORIBA LabRAM HR Evolution Confocal Raman instrument. The system was equipped with a high spatial-resolution confocal microscope, a high performance Raman spectrometer multiple detectors, and used high-precision DuoScan imaging technology in order to enhance the measured S/N ratios and precision. The excitation light source in the measurements was a 785 nm laser, and the scan range was from 125 to 1000 cm⁻¹.

### (4) Powder X-Ray Diffraction Analysis

Powder X-ray diffraction (PXRD) analyses were carried out on a STOE-STADI MP powder diffractometer equipped with an asymmetric curved Germanium monochromator (Cu-Kα1 radiation, λ=1.54056 Å) and a one-dimension silicon strip detector (MYTHEN2 1K from DECTRIS). Samples for structural analysis were measured at room temperature in transmission geometry. The simulated PXRD patterns were calculated using the Mercury software 4.3.0.

### (5) Inductively Coupled Plasma Optical Emission Spectrometry Analysis

Inductively coupled plasma optical emission spectrometry (ICP-OES) analyses were performed on a thermo iCap7600 ICP-OES (Thermo Fisher Scientific, Waltham, MA, USA) operating in radial view and equipped with a CETAC 520 autosampler (Omaha, NE, USA). The samples were filtered through a 0.45-μm filter. The filtrates were then diluted with ultrapure H₂O and analyzed for the concentration of Pt, Pd, and Rh in comparison with standard solutions. Each sample was recorded using 5 sec visible exposure and 15 sec UV exposure time. Every sample was measured repeatedly for 3 times. The wavelengths selected for the analyses of the concentration of Pt were 203.646, 214.423, and 265.945 nm. The wavelengths selected for the analyses of the concentration of Pd were 324.270, 340.458, and 360.955 nm. The wavelengths selected for the analyses of the concentration of Rh were 339.682, 343.489, and 369.236 nm.

### (6) X-Ray Photoelectron Spectroscopy

The X-ray photoelectron spectral (XPS) analyses were conducted on a fully digital state-of-the-art X-ray photoelectron spectrometer (Thermo Scientific ESCALAB 250Xi), which was equipped with an electron flood gun and a scanning ion gun. The diameter of X-ray spot was 500 μm, and the scan range was from 0 to 1200 eV.

### General Methods

### (1) Co-Crystallization Between CB[6] and Pt, Pd and Rh Chlorides

Aqueous stock solutions of H₂PtCl₆, (NH₄)₂PtCl₄, cisplatin, transplatin, (NH₄)₂PdCl₆, (NH₄)₂PdCl₄, (NH₄)₃RhCl₆ were prepared by dissolving directly the corresponding commercially available complexes in high purity H₂O, while aqueous solutions of CB[6] were prepared by dissolving CB[6] in an aqueous HCl (4 M) solution. When an CB[6] (1 mL, 10 mM) aqueous solution was added to the H₂PtCl₆, (NH₄)₂PtCl₄, cisplatin, transplatin, (NH₄)₂PdCl₆, (NH₄)₂PdCl₄, (NH₄)₃RhCl₆ aqueous solutions, respectively, a yellow suspension was formed specifically between CB[6] and H₂PtCl₆. The yellow solids were isolated by filtration, washing, and air-dried. The concentrations of [PtCl₆]²⁻, [PdCl₄]²⁻, and [RhCl₆]³⁻ remaining in the filtrates were determined by ICP-OES analysis. The metal precipitation yields were calculated based on the initial and residue concentrations of metal anions in the aqueous solution.

### (2) Platinum Recovery and CB[6] Regeneration

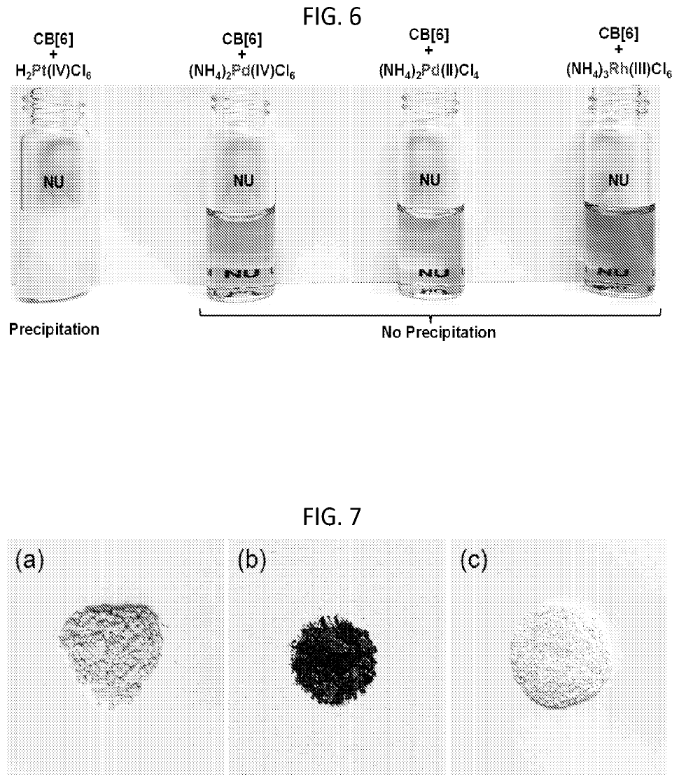

When an aqueous solution of CB[6] (1.0 mL, 10 mM) was mixed with equimolar amounts of [PtCl₆]²⁻, [PdCl₄]²⁻ and [RhCl₆]³⁻ anions, a yellow suspension formed immediately. The yellow solid (116.7 mg, FIG. 7 (a)) was isolated by filtration, washing, and air drying. Subsequently, the yellow solid was dispersed in H₂O (4 mL), and reduced by 78% N₂H₄·H₂O (1.0 mL). A black suspension was obtained after heating at 60° C. for 2 h with stirring. Following centrifugation and washing with 4 M HCl aqueous solution three times to dissolve the residual CB[6], black platinum metal (14.1 mg, FIG. 7 (b)) was obtained. The purity of the recovered platinum was found (Table 8) to be 94.9%. The CB[6] that remain dissolved in the aqueous solution can be recycled by precipitating with Me₂CO. After centrifugation and washing with H₂O and Me₂CO three times, the regenerated CB[6] (91.0 mg, FIG. 7 (c)) was isolated as a white powder. The regenerated CB[6] can be used (Table 1) in subsequent rounds of platinum recovery.

TABLE 1
The Pt-Recovery Efficiency over Three Precipitation/Pt
Reduction/CB[6] Regeneration Cycles

| Cycles | First | Second | Third |
|---|---|---|---|
| Pt-Precipitation | 83.0 | 80.4 | 76.8 |
| Yields/% | | | |
| Pt-Reduction | 87.1 | 89.3 | 91.4 |
| Yields/% | | | |
| Total Pt-Recovery | 72.3 | 71.8 | 70.2 |
| Efficiencies/% | | | |

### Crystallographic Characterization

(1) CB[6]·H₂PtCl₆
(a) Method. Stock aqueous solutions of H₂PtCl₆ (200 μL, 10 mM) were layered carefully upon an aqueous CB[6] (200 μL, 10 mM) solution with 4 M HCl in 1-mL tubes. High quality yellow cocrystals of CB[6]·H₂PtCl₆ were obtained by liquid-liquid diffusion for 6 h. Suitable crystals were selected and mounted on a MITIGEN holder with Paratone oil on an XtaLAB Synergy, Single source at offset/far, HyPix diffractometer. The crystals were kept at ˜100 K during the data collection. Using Olex2,² the structures were solved with the ShelXT³ structure solution program using Intrinsic Phasing and refined with the XL⁴ refinement package employing Least Squares Minimization. Crystallographic images were produced using Mercury 4.3.0. Distances were measured employing Mercury 4.3.0. The solid-state (super)structure of CB[6]·H₂PtCl₆ is shown in FIG. 3.
(b) Crystal Parameters. C₃₆H₃₆N₂₄O₁₂·H₂PtCl₆·9(H₂O). Mr=1568.83. Yellow block (0.416×0.069×0.014 mm³). Monoclinic, space group I2/a (no. 15), a=13.3214(3), b=20.6716(5), c=24.2395(5) Å, α=90.000, β=94.433(2), γ=90.000°, V=6655.0(2) Å³, Z=4, T=100.0(2) K, μ(MoKα)=2.432 mm⁻¹, Dcalc=1.566 g/mm³, 47648 reflections measured (5.124≤2Θ≤67.59), 11762 unique (Rᵢₙₜ=0.0408, R_{sigma}=0.0423) which were used in all calculations. The final R₁ was 0.0401 (I>2σ(I)) and wR₂ was 0.1099 (all data).
(c) Refinement and solvent treatment details. No special refinement was necessary in the case of solving the solid-state superstructure of CB[6]·H₂PtCl₆. The solvent-masking procedure as implemented in Olex2 was used in order to remove the electronic contribution of solvent molecules from the refinement. As the exact solvent content is not known, only the atoms used in the refinement model and protons that were added for charge balance are reported in the formula here. Total solvent accessible volume/cell=1305.5 Å³ [19.6%], Total electron count/cell=378.6.
   (2) CB[6]·(NH₄)₂PtCl₄
(a) Method. An equimolar amount of (NH₄)₂PtCl₄ (500 μL, 10 mM) was added to an aqueous CB[6] (500 μL, 10 mM) solution with 4 M HCl in a 3-mL vial. High quality yellow cocrystals of CB[6]·H₂PtCl₄ were obtained after slow evaporation for one week. Suitable crystals were selected and mounted on a MITIGEN holder with Paratone oil on an XtaLAB Synergy, DW system, HyPix diffractometer. The crystals were kept at 103 K during the data collection. Using Olex2,² the structures were solved with the ShelXT³ structure solution program using Intrinsic Phasing and refined with the XL⁴ refinement package employing Least Squares Minimization. Crystallographic images were produced using Mercury 4.3.0. Distances were measured employing Mercury 4.3.0. The solid-state (super)structure of CB[6]·H₂PtCl₄ is shown in FIG. 8.
(b) Crystal Parameters. 3(C₃₆H₃₆N₂₄O₁₂)·2(H₂PtCl₄)·14(H₂O). Mr=3920.69. Yellow block (0.805×0.481×0.201 mm³). Monoclinic, space group P2₁/c (no. 14), a=16.39259(8), b=14.34159(7), c=35.65622(17) Å, α=90.000, β=98.3483(4), γ=90.000°, V=8293.80(7) Å³, Z=2, T=102.8(8) K, (CuKα)=5.165 mm⁻¹, Dcalc=1.570 g/mm³, 160793 reflections measured (5.01≤2Θ≤157.116), 17608 unique (Rᵢₙₜ=0.0602, R_{sigma}=0.0248) which were used in all calculations. The final R₁ was 0.0518 (I>2σ(I)) and wR₂ was 0.1491 (all data).
(c) Refinement and solvent treatment details. Enhanced rigid-bond restraint⁵ was applied globally. The solvent-masking procedure as implemented in Olex2 was used in order to remove the electronic contribution of solvent molecules from the refinement. As the exact solvent content is not known, only the atoms used in the refinement model and protons that were added for charge balance are reported in the formula here. Total solvent accessible volume/cell=1720.3 Å³ [20.7%], Total electron count/cell=589.6.
   (3) CB[6]·(NH₄)₂PdCl₄
(a) Method. An equimolar amount of (NH₄)₂PdCl₄ (500 μL, 10 mM) was added to an aqueous CB[6] (500 μL, 10 mM), solution with 4 M HCl in a 3-mL vial. High quality yellow cocrystals of CB[6]·H₂PdCl₄ were obtained after slow evaporation for one week. Suitable crystals were selected and mounted on a MITIGEN holder with Paratone oil on an XtaLAB Synergy, Single source at offset/far, HyPix diffractometer. The crystals were kept at 100 K during the data collection. Using Olex2,² the structures were solved with the ShelXT³ structure solution program using Intrinsic Phasing and refined with the XL⁴ refinement package employing Least Squares Minimization. Crystallographic images were produced using Mercury 4.3.0. Distances were measured employing Mercury 4.3.0. The solid-state (super)structure of CB[6]·H₂PdCl₄ is shown in FIG. 9.
(b) Crystal Parameters. 1.5(C₃₆H₃₆N₂₄O₁₂)·H₂PdCl₄·6.5(H₂O). Mr=1862.65. Yellow block (0.805×0.481×0.201 mm³). Monoclinic, space group P2₁/c (no. 14), a=16.3624(2), b=14.3331(2), c=35.7320(7) Å, α=90.000, β=98.327(2), γ=90.000°, V=8291.7(2) Å³, Z=4, T=100.00(10) K, (MoKα)=0.446 mm⁻¹, Dcalc=1.492 g/mm³, 140047 reflections measured (4.07≤2Θ≤67.794), 29347 unique (Rᵢₙₜ=0.0370, R_{sigma}=0.0367) which were used in all calculations. The final R₁ was 0.0827 (I>2σ(I)) and wR₂ was 0.2836 (all data).
(c) Refinement and solvent treatment details. No special refinement was necessary in the case of solving the solid-state superstructure of CB[6]·H₂PdCl₄. The solvent-masking procedure as implemented in Olex2 was used in order to remove the electronic contribution of solvent molecules from the refinement. As the exact solvent content is not known, only the atoms used in the refinement model and protons that were added for charge balance are reported in the formula here. Total solvent accessible volume/cell=1739.9 Å³ [21.0%], Total electron count/cell=489.4.
   (4) CB[6]·(NH₄)₃RhCl₆
(a) Method. An equimolar amount of (NH₄)₃RhCl₆ (500 μL, 10 mM) was added to an aqueous CB[6] (500 μL, 10 mM) solution with 4 M HCl in a 3-mL vial. High quality red cocrystals of CB[6]·H₃RhCl₆ were obtained after slow evaporation for one week. Suitable crystals were selected and mounted on a MITIGEN holder with Paratone oil on an XtaLAB Synergy, DW system, HyPix diffractometer. The crystals were kept at 100 K during the data collection. Using Olex2,² the structures were solved with the ShelXT³ structure solution program using Intrinsic Phasing and refined with the XL⁴ refinement package employing Least Squares Minimization. Crystallographic images were produced using Mercury 4.3.0. Distances were measured employing Mercury 4.3.0. The solid-state (super)structure of CB[6]·H₃RhCl₆ is shown in FIG. 10.
(b) Crystal Parameters. 3(C₃₆H₃₆N₂₄O₁₂)·H₃RhCl₆·12(H₂O). Mr=3525.48. Red block (0.146×0.107×0.072 mm³). Trigonal, space group R3̅ (no. 148), a=32.0478(5), b=32.0478(5), c=12.4461(3) Å, α=90.000, β=90.000, γ=120.000°, V=11070.3(4) Å³, Z=3, T=99.97(10) K, (CuKα)=2.888 mm⁻¹, Dcalc=1.586 g/mm³, 23763 reflections measured (5.516≤2Θ≤157.07), 5117 unique (Rᵢₙₜ=0.0260, R_{sigma}=0.0169) which were used in all calculations. The final R₁ was 0.0796 (I>2σ(I)) and wR₂ was 0.2254 (all data).
(c) Refinement and solvent treatment details. No special refinement was necessary in the case of solving the solid-state superstructure of CB[6]·H₃RhCl₆. The solvent-masking procedure as implemented in Olex2 was used in order to remove the electronic contribution of solvent molecules from the refinement. As the exact solvent content is not known, only the atoms used in the refinement model and protons that were added for charge balance are reported in the formula here. Total solvent accessible volume/cell=794.1 Å³ [7.2%], Total electron count/cell=342.3.

TABLE 2
Crystallographic Data for Four Adducts Between CB[6] and Pt, Pd and Rh Chlorides

| Complex | CB[6]•H₂PtCl₆ | CB[6]•(NH₄)₂PtCl₄ | CB[6]•(NH₄)₂PdCl₄ |
|---|---|---|---|
| Empirical | C₃₆H₃₆N₂₄O₁₂•H₂PtCl₆•9(H₂O) | 3(C₃₆H₃₆N₂₄O₁₂)•2(H₂PtCl₄)•14(H₂O) | 1.5(C₃₆H₃₆N₂₄O₁₂)•H₂PdCl₄•6.5(H₂O) |
| formula | | | |
| Formula | 1568.83 | 3920.69 | 1862.65 |
| weight | | | |
| T/K | 100.0(2) | 102.8(8) | 100.00(10) |
| Crystal | monoclinic | monoclinic | monoclinic |
| system | | | |
| Space | I2/a | P2₁/c | P2₁/c |
| group | | | |
| a/Å | 13.3214(3) | 16.39259(8) | 16.3624(2) |
| b/Å | 20.6716(5) | 14.34159(7) | 14.3331(2) |
| c/Å | 24.2395(5) | 35.65622(17) | 35.7320(7) |
| α/° | 90 | 90 | 90 |
| β/° | 94.433(2) | 98.3483(4) | 98.327(2) |
| γ/° | 90 | 90 | 90 |
| V/Å³ | 6655.0(2) | 8293.80(7) | 8291.7(2) |
| Z | 4 | 2 | 4 |
| ρ_{calcd}/g | 1.566 | 1.570 | 1.492 |
| cm⁻³ | | | |
| μ/mm⁻¹ | 2.432 | 5.165 | 0.446 |
| F (000) | 3144 | 3960 | 3812 |
| goodness- | 1.041 | 1.036 | 1.053 |
| of-fit on F² | | | |
| Rₗ [I > 2σ | 0.0401 | 0.0518 | 0.0827 |
| (I)] | | | |
| wR₂ [all | 0.1099 | 0.1491 | 0.2836 |
| data] | | | |

| Complex | CB[6]•(NH₄)₃RhCl₆ |
|---|---|
| Empirical | 3(C₃₆H₃₆N₂₄O₁₂)•H₃RhCl₆•12(H₂O) |
| formula | |
| Formula | 3525.48 |
| weight | |
| T/K | 99.97(10) |
| Crystal | trigonal |
| system | |
| Space | R3̅ |
| group | |
| a/Å | 32.0478(5) |
| b/Å | 32.0478(5) |
| c/Å | 12.4461(3) |
| α/° | 90 |
| β/° | 90 |
| γ/° | 120 |
| V/Å³ | 11070.3(4) |
| Z | 3 |
| ρ_{calcd}/g | 1.586 |
| cm⁻³ | |
| μ/mm⁻¹ | 2.888 |
| F (000) | 5445 |
| goodness- | 1.118 |
| of-fit on F² | |
| Rₗ [I > 2σ | 0.0796 |
| (I)] | |
| wR₂ [all | 0.2254 |
| data] | |

### ICP-OES Analysis

Effect of Changes in Concentration of CB[6]·H₂PtCl₆ on the Pt-Precipitation Yields

TABLE 3
Changes in Pt-Precipitation Yields with Respect to the Concentration
of CB[6]•H₂PtCl₆ in 2M HCl Aqueous Solutions

| Concentration | | | | | | | |
|---|---|---|---|---|---|---|---|
| of | Pt | Pt | Pt | Avg. | Pt in | | |
| CB[6]•H₂PtCl₆/ | 214.423/ | 265.945/ | 203.646/ | Pt/ | Filtrate/ | Total Pt/ | |
| mM | ppm | ppm | ppm | ppm | mg | mg | Yields |
| 10.0 | 4.911 | 4.888 | 4.878 | 4.892 | 0.294 | 1.214 | 0.758 |
| 8.0 | 3.612 | 3.708 | 3.580 | 3.634 | 0.273 | 1.214 | 0.776 |
| 6.0 | 2.331 | 2.383 | 2.302 | 2.338 | 0.117 | 0.607 | 0.807 |
| 5.0 | 2.011 | 2.049 | 1.989 | 2.016 | 0.121 | 0.607 | 0.801 |
| 4.0 | 1.928 | 1.920 | 1.892 | 1.913 | 0.143 | 0.607 | 0.764 |
| 2.0 | 1.063 | 1.050 | 1.050 | 1.054 | 0.158 | 0.607 | 0.739 |

Effect of Changes in Concentration on the Pt/Pd/Rh-Precipitation Yields from Mixture

TABLE 4
Changes in Pt-Precipitation Yields with Respect to the Concentration
of Pt, Pd, Rh Mixtures in 2M HCl Aqueous Solutions

| Concentration | Pt | Pt | Pt | Avg. | Pt in | | |
|---|---|---|---|---|---|---|---|
| of Mixture/ | 214.423/ | 265.945/ | 203.646/ | Pt/ | Filtrate/ | Total Pt/ | |
| mM | ppm | ppm | ppm | ppm | mg | mg | Yields |
| 10.0 | 4.981 | 4.974 | 4.919 | 4.958 | 0.297 | 1.192 | 0.750 |
| 8.0 | 3.961 | 3.974 | 3.907 | 3.947 | 0.296 | 1.192 | 0.752 |
| 6.0 | 2.326 | 2.342 | 2.294 | 2.320 | 0.116 | 0.596 | 0.805 |
| 5.0 | 2.088 | 2.038 | 2.052 | 2.059 | 0.124 | 0.596 | 0.793 |
| 4.0 | 1.800 | 1.743 | 1.770 | 1.771 | 0.133 | 0.596 | 0.777 |
| 2.0 | 1.188 | 1.173 | 1.183 | 1.182 | 0.177 | 0.596 | 0.703 |

TABLE 5
Changes in Pd-Precipitation Yields with Respect to the Concentration
of Pt, Pd, Rh Mixtures in 2M HCl Aqueous Solutions

| Concentration | Pd | Pd | Pd | Avg. | Pd in | | |
|---|---|---|---|---|---|---|---|
| of Mixture/ | 340.458/ | 324.270/ | 360.955/ | Pd/ | Filtrate/ | Total Pd/ | |
| mM | ppm | ppm | ppm | ppm | mg | mg | Yields |
| 10.0 | 8.271 | 9.699 | 9.527 | 9.166 | 0.550 | 0.569 | 0.033 |
| 8.0 | 6.652 | 7.819 | 7.651 | 7.374 | 0.553 | 0.569 | 0.028 |
| 6.0 | 4.732 | 5.547 | 5.401 | 5.227 | 0.261 | 0.285 | 0.083 |
| 5.0 | 3.997 | 4.732 | 4.650 | 4.460 | 0.268 | 0.285 | 0.061 |
| 4.0 | 3.157 | 3.760 | 3.697 | 3.538 | 0.265 | 0.285 | 0.069 |
| 2.0 | 1.584 | 1.883 | 1.810 | 1.759 | 0.264 | 0.285 | 0.074 |

TABLE 6
Changes in Rh-Precipitation Yields with Respect to the Concentration
of Pt, Pd, Rh Mixtures in 2M HCl Aqueous Solutions

| Concentration | Rh | Rh | Rh | Avg. | Rh in | | |
|---|---|---|---|---|---|---|---|
| of Mixture/ | 343.489/ | 369.236/ | 339.682/ | Rh/ | Filtrate/ | Total Rh/ | |
| mM | ppm | ppm | ppm | ppm | mg | mg | Yields |
| 10.0 | 9.326 | 9.145 | 8.332 | 8.934 | 0.536 | 0.538 | 0.004 |
| 8.0 | 7.459 | 7.294 | 6.687 | 7.147 | 0.536 | 0.538 | 0.004 |
| 6.0 | 5.543 | 5.415 | 4.942 | 5.300 | 0.265 | 0.269 | 0.015 |
| 5.0 | 4.615 | 4.502 | 4.160 | 4.426 | 0.266 | 0.269 | 0.013 |
| 4.0 | 3.630 | 3.576 | 3.192 | 3.466 | 0.260 | 0.269 | 0.034 |
| 2.0 | 1.842 | 1.790 | 1.614 | 1.748 | 0.262 | 0.269 | 0.025 |

TABLE 7
Pt-Precipitation Yield Based on the CB[6]•
(NH₄)₂PtCl₆ Cocrystals in 2M HCl Aqueous Solutions

| Concentration of | Pt | Pt | Pt | Avg. | Pt in | | |
|---|---|---|---|---|---|---|---|
| CB[6]•(NH₄)₂PtCl₆/ | 214.423/ | 265.945/ | 203.646/ | Pt/ | Filtrate/ | Total Pt/ | |
| mM | ppm | ppm | ppm | ppm | mg | mg | Yield |
| 5.0 | 1.560 | 1.630 | 1.560 | 1.584 | 0.095 | 0.603 | 0.842 |

TABLE 8
The Purity of Recovered Platinum

| | Pt | Pt | Pt | Avg. | Pt in | Recovered | |
|---|---|---|---|---|---|---|---|
| | 214.423/ | 265.945/ | 203.646/ | Pt/ | solution/ | Pt/ | |
| | ppm | ppm | ppm | ppm | mg | mg | Purity |
| Recovered | 2.252 | 2.331 | 2.249 | 2.277 | 2.28 | 2.40 | 0.949 |
| Platinum | | | | | | | |

### Density Function Theory Calculations

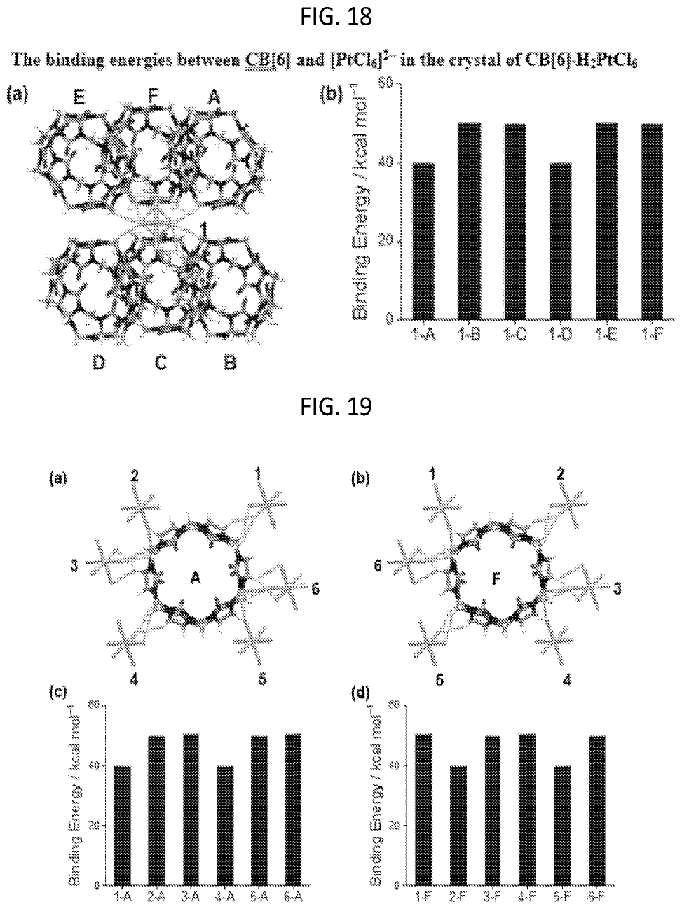

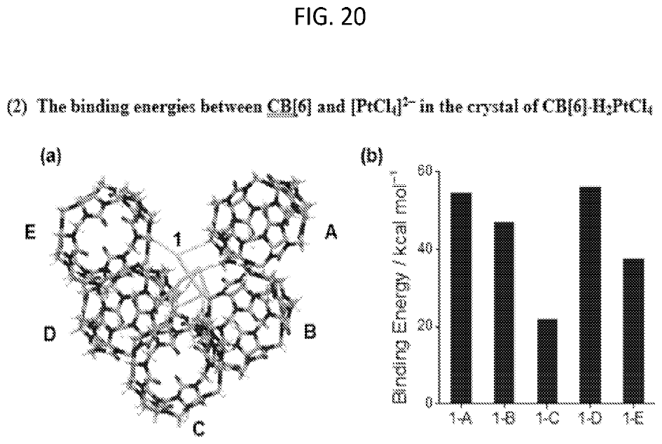

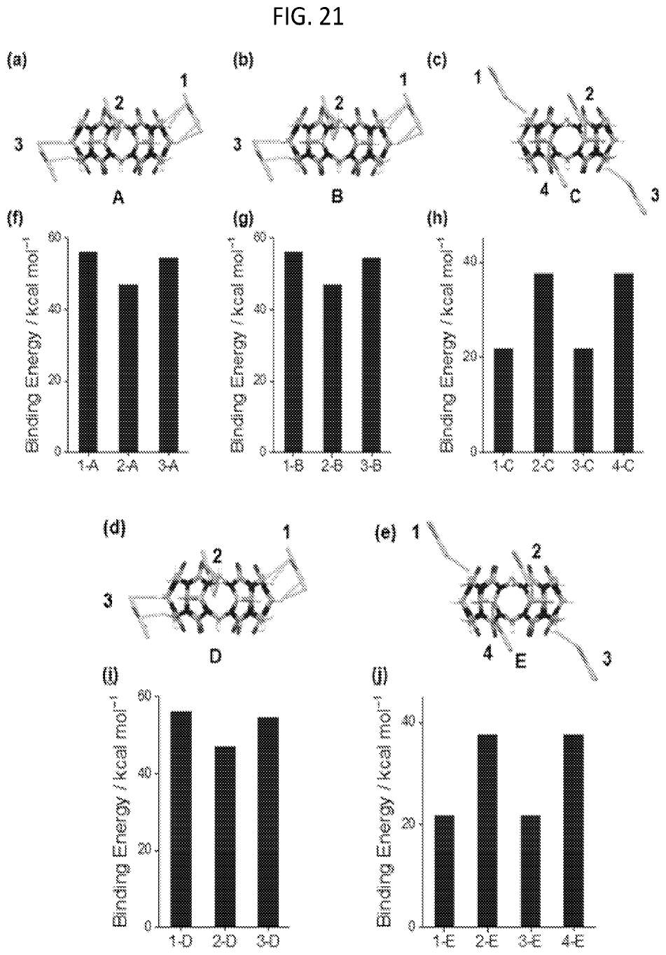

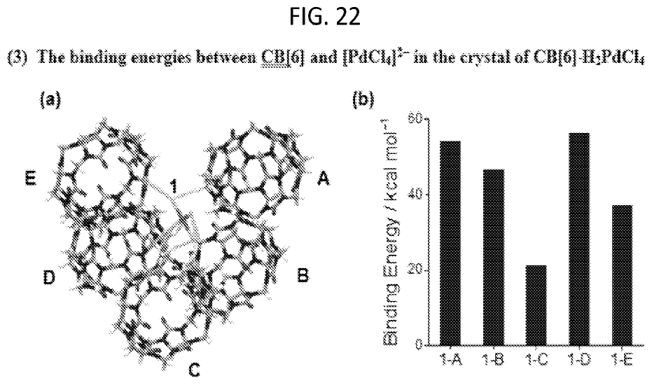

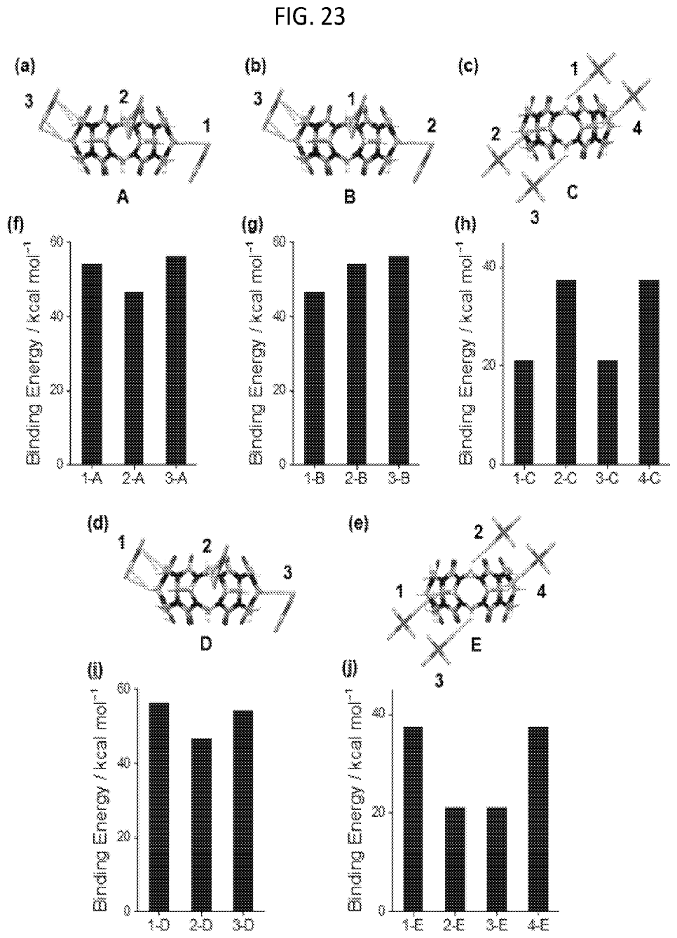

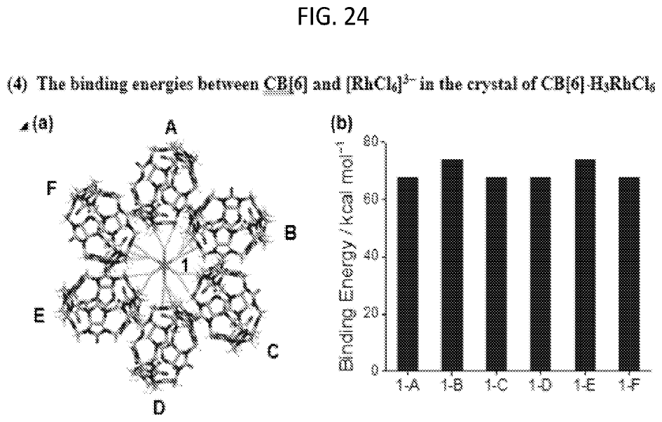

In order to gain a better understanding of the noncovalent bonding interactions and binding energies between CB[6] molecules and [PtCl₆]²⁻, [PtCl₄]²⁻, [PdCl₄]²⁻ and [RhCl₆]³⁻ anions, as well as the adjacent CB[6] molecules, density function theory (DFT) calculations have been carried out based on the crystal superstructures of CB[6]·H₂PtCl₆, CB[6]·H₂PtCl₄, CB[6]·H₂PdCl₄, and CB[6]·H₃RhCl₆. FIGS. 18-19 show the binding modes and binding energies between CB[6] molecules and [PtCl₆]²⁻ dianions. FIGS. 20-21 show the binding modes and binding energies between CB[6] molecules and [PtCl₄]²⁻ dianions. FIGS. 22-23 show the binding modes and binding energies between CB[6] molecules and [PdCl₄]²⁻ dianions. FIGS. 24-25 show the binding modes and binding energies between CB[6] molecules and [RhCl₆]³⁻ trianions. Table 9 shows the total binding energies between four anions with their adjacent CB[6] molecules. FIG. 26 shows the binding modes and binding energies between adjacent CB[6] molecules of four adducts.

The xyz coordinates for the single point calculations were extracted from the single-crystal X-ray crystallographic data of the four adducts. Single point calculations were performed with two levels of density functional theory (DFT) in the Orca program⁶ (version 4.1.2). (1) For the majority of binding energies, the hybrid Becke three-parameter Lee-Yang-Parr⁷ (B3LYP) functional, the Ahlrich's double zeta basis set with a polarization function⁸ Def2-SVP, Grimme's third generation dispersion with Beck Johnson damping (D3BJ), and an integration grid of 4 (Grid4) were used to evaluate the electronic energy. (2) For the binding energy of one anion with the nearest CB[6] neighbors computed as a single cluster, a lower less memory intensive level of pure Hartree-Fock with the Ahlrich's double zeta, reduced polarization functions basis Def2-SV(P), and default integration grid (Grid2) was used, without dispersion. In order to speed up the single point calculations with B3LYP, the Coulomb integral and numerical chain-of-sphere integration for the HF exchange^{9,10} (RIJCOSX) method was applied with the Def2/J auxiliary basis¹¹ (AuxJ).

TABLE 9
Results of DFT Calculation for the Total Binding Energies
Between One [PtCl₆]²⁻, [PtCl₄]²⁻, [PdCl₄]²⁻ and [
RhCl₆]³⁻ and Their Connected CB[6] in the
Solid State, Respectively

| Entry | E_{b}/kcal mol⁻¹ |
|---|---|
| [PtCl₆]²⁻. . . 6CB[6] | 136.38 |
| [PtCl₄]²⁻ . . . 5CB[6] | 113.00 |
| [PdCl₄]²⁻ .. . 5CB[6] | 115.40 |
| [RhCl₆]³⁻ . . . 6CB[6] | 226.70 |

Visualization of Noncovalent Bonding Interactions Between CB[6] and H₂PtCl₆ in Solid-State Superstructure

Independent gradient model (IGM) analysis is an approach based on promolecular density—an electron density model prior to molecule formation—to identify and isolate intermolecular interactions. Strong polar attractions and van der Waals contacts are visualized as an iso-surface, respectively. Single crystal superstructures were used as input files. The binding surface was calculated using the Multiwfn 3.6 program through function 20 (visual study of weak interaction) and visualized by Chimera software.

### REFERENCES

[1] a) J. Kim, I. S. Jung, S. Y. Kim, E. Lee, J. K. Kang, S. Sakamoto, K. Yamaguchi, K. Kim, J. Am. Chem. Soc. 2000, 122, 540-541; b) A. Day, A. P. Arnold, R. J. Blanch, B. Snushall, J. Org. Chem. 2001, 66, 8094-8100.
[2] O. V. Dolomanov, L. J. Bourhis, R. J. Gildea, J. A. K. Howard, H. Puschmann, J. Appl. Cryst. 2009, 42, 339-341.
[3] G. M. Sheldrick, Acta. Cryst. 2015, A71, 3-8.
[4] G. M. Sheldrick, Acta. Cryst. 2008, A64, 112-122.
[5] A. Thorn, B. Dittrich, G. M. Sheldrick, Acta. Cryst. 2012, A68, 448-451.
[6] F. Neese, Wiley Interdiscip. Rev.: Comput. Mol. Sci. 2012, 2, 73-78.
[7] A. D. Becke, J. Chem. Phys. 1993, 98, 5648-5652.
[8] F. Weigend, R. Ahlrichs, Phys. Chem. Chem. Phys. 2005, 7, 3297-3305.
[9] F. Neese, J. Comp. Chem. 2003, 24, 1740-1747.
[10] R. Izsik, F. Neese, J. Chem. Phys. 2011, 135, 144105.
[11] a) F. Weigend, Phys. Chem. Chem. Phys. 2006, 8, 1057-1065; b) G. L. Stoychev, A. A. Auer, F. Neese, J. Theo. Comp. Chem. 2017, 13, 554-562.
[12] C. Lefebvre, G. Rubez, H. Khartabil, J. C. Boisson, J. Contreras-Garcia, E. Hénon, Phys. Chem. Chem. Phys. 2017, 19, 17928-17936.
[13] T. Lu, F. Chen, J. Comput. Chem. 2012, 33, 580-592.
[14] E. F. Pettersen, T. D. Goddard, C. C. Huang, G. S Couch, D. M. Greenblatt, E. C. Meng, T. E. Ferrin, J. Comput. Chem. 2004, 25, 1605-1612.

1. An adduct comprising a platinum halide dianion non-covalently bound to the outer surface of a cucurbituril.

2. The adduct of claim 1, wherein the platinum halide dianion is [PtX₆]²⁻ and X is a halogen.

3. The adduct of claim 1, wherein the platinum halide dianion is [PtCl₆]²⁻.

4. The adduct of claim 1, wherein the cucurbituril is cucurbitu[6]ril.

5. The adduct of claim 1, wherein the adduct comprises the cucurbitu[6]ril and [PtCl₆]²⁻, wherein the platinum halide dianion is non-covalently bound by a [Pt—Cl . . . H—C]hydrogen bond and/or a [Pt—Cl . . . C═O] ion-dipole interaction.

6. A superstructure comprising the adduct of claim 1.

7. The superstructure of claim 6, wherein the molar ratio of the platinum halide dianion and cucurbituril is 1:1.

8. The superstructure of claim 7, wherein the cucurbituril is surrounded by six adjacent platinum halide dianions.

9. The superstructure of claim 7, wherein every platinum halide dianion is surrounded by six cucurbituril molecules and stabilized by 14 sets of hydrogen bonds and two sets of ion-dipole interactions.

10. A crystalline composition comprising the adduct of claim 1.

11. The crystalline composition of claim 10, wherein the crystalline composition is in the monoclinic space group I2/a.

12. The crystalline composition of claim 11, wherein the crystalline composition has lattice parameters of a=13.3±0.2 Å, b=20.7±0.2 Å, c=24.2±0.2 Å, α=90.0±0.5°, β=94.4±0.5°, and γ=90.0±0.5°.

13. A method for separation of a metal, the method comprising contacting a platinum halide dianion with a cucurbitrurial under conditions sufficient to cause co-precipitation of the platinum metal dianion and the cucurbitrurial.

14. The method of claim 13, wherein cucurbituril is cucurbitu[6]ril and the halide dianion is [PtX₆]²⁻ and X is a halogen, optionally wherein the platinum metal dianion is [PtCl₆]²⁻.

15. The method of claim 13, further comprising contacting a platinum-bearing material with a hydrogen halide to form a metal-halide solution comprising the platinum halide dianion and wherein the platinum halide dianion in the metal-halide solution is contacted with the cucurbitrurial.

16. The method of claim 13, further comprising reducing platinum of the precipitate with a reductant.

17. The method of claim 16, further comprising isolating the reduced platinum.

18. The method of claim 13, further comprising isolating the cucurbitrurial of the precipitate.

19. The method of claim 18, wherein the isolated cucurbitrurial is recycled.

20. The method of claim 13, wherein the precipitate comprises an adduct comprising a platinum halide dianion non-covalently bound to the outer surface of a cucurbituril, a superstructure comprising the adduct, or a crystalline composition comprising the adduct.
